(19) United States
(12) Patent Application Publication
(10) Pub. No.: US 2021/0186752 A1
(43) Pub. Date: Jun. 24, 2021

(54) SYSTEM AND METHOD FOR LOCATING A STRUCTURE OF OCULAR TISSUE FOR GLAUCOMA SURGERY BASED ON SECOND HARMONIC LIGHT

(71) Applicant: ViaLase, Inc., Aliso Viejo, CA (US)

(72) Inventors: Tibor Juhasz, San Clemente, CA (US); Eric R. Mikula, Aliso Viejo, CA (US)

(21) Appl. No.: 16/723,883

(22) Filed: Dec. 20, 2019

(51) Int. Cl. A61F 9/008

(52) U.S. Cl. CPC A61F 9/008; A61F 2009/00891; A61F 2009/0088; A61F 2009/00851

(57) ABSTRACT

A structure in an irido-corneal angle of an eye is located by directing a laser beam toward the irido-corneal angle of the eye, and advancing a focus of the laser beam to a location in the irido-corneal angle, which location is at or near the target structure. The focus is determined to be at or near the structure based on changes in an intensity of a spot of second harmonic light generated by an encounter between the focus and tissue.

### TECHNICAL FIELD

The present disclosure relates generally to the field of medical devices and treatment of diseases in ophthalmology including glaucoma, and more particularly to systems and methods for locating a structure of ocular tissue based on second harmonic light.

### BACKGROUND

Before describing the different types of glaucoma and current diagnosis and treatments options, a brief overview of the anatomy of the eye is provided.

Anatomy of the Eye

With reference to FIGS. 1-3, the outer tissue layer of the eye 1 includes a sclera 2 that provides the structure of the eye's shape. In front of the sclera 2 is a cornea 3 that is comprised of transparent layers of tissue that allow light to enter the interior of the eye. Inside the eye 1 is a crystalline lens 4 that is connected to the eye by fiber zonules 5, which are connected to the ciliary body 6. Between the crystalline lens 4 and the cornea 3 is an anterior chamber 7 that contains a flowing clear liquid called aqueous humor 8. Encircling the perimeter of the crystalline lens 4 is an iris 9 which forms a pupil around the approximate center of the crystalline lens. A posterior chamber 23 is an annular volume behind the iris 9 and bounded by the ciliary body 6, fiber zonules 5, and the crystalline lens 4. The vitreous humor 10 is located between the crystalline lens 4 and the retina 11. Light entering the eye is optically focused through the cornea 3 and crystalline lens.

With reference to FIG. 2, the corneoscleral junction of the eye is the portion of the anterior chamber 7 at the intersection of the iris 9, the sclera 2, and the cornea 3. The anatomy of the eye 1 at the corneoscleral junction includes a trabecular meshwork 12. The trabecular meshwork 12 is a fibrous network of tissue that encircles the iris 9 within the eye 1. In simplified, general terms the tissues of the corneoscleral junction are arranged as follows: the iris 9 meets the ciliary body 6, the ciliary body meets with the underside of the scleral spur 14, the top of the scleral spur serves as an attachment point for the bottom of the trabecular meshwork 12. The ciliary body is present mainly in the posterior chamber, but also extends into the very corner of the anterior chamber 7. The network of tissue layers that make up the trabecular meshwork 12 are porous and thus present a pathway for the egress of aqueous humor 8 flowing from the anterior chamber 7. This pathway may be referred to herein as an aqueous humor outflow pathway, an aqueous outflow pathway, or simply an outflow pathway.

Referring to FIG. 3, the pathway formed by the pores in the trabecular meshwork 12 connect to a set of thin porous tissue layers called the uveal meshwork 15, the corneoscleral meshwork 16, and the juxtacanalicular tissue 17. The juxtacanalicular tissue 17, in turn, abuts a structure called Schlemm's canal 18. The Schlemm's canal 18 carries a mixture of aqueous humor 8 and blood from the surrounding tissue to drain into the venous system though a system of collector channels 19. As shown in FIG. 2, the vascular layer of the eye, referred to as the choroid 20, is next to the sclera 2. A space, called the suprachoroidal space 21, may be present between the choroid 20 and the sclera 2. The general region near the periphery of the wedge between the cornea 3 and the iris 9, running circumferentially is called the irido-corneal angle 13. The irido-corneal angle 13 may also be referred to as the corneal angle of the eye or simply the angle of the eye. The ocular tissues illustrated in FIG. 3 are all considered to be within the irido-corneal angle 13.

With reference to FIG. 4, two possible outflow pathways for the movement of aqueous humor 8 include a trabecular outflow pathway 40 and a uveoscleral outflow pathway 42. Aqueous humor 8, which is produced by the ciliary body 6, flows from the posterior chamber 23 through the pupil into the anterior chamber 7, and then exits the eye through one or more of the two different outflow pathways 40, 42. Approximately 90% of the aqueous humor 8 leaves via the trabecular outflow pathway 40 by passing through the trabecular meshwork 12, into the Schlemm's canal 18 and through one or more plexus of collector channels 19 before draining through a drain path 41 into the venous system. Any remaining aqueous humor 8 leaves primarily through the uveoscleral outflow pathway 42. The uveoscleral outflow pathway 42 passes through the ciliary body 6 face and iris root into the suprachoroidal space 21 (shown in FIG. 2). Aqueous humor 8 drains from the suprachoroidal space 21, from which it can be drained through the sclera 2.

The intra-ocular pressure of the eye depends on the aqueous humor 8 outflow through the trabecular outflow pathway 40 and the resistance to outflow of aqueous humor through the trabecular outflow pathway. The intra-ocular pressure of the eye is largely independent of the aqueous humor 8 outflow through the uveoscleral outflow pathway 42. Resistance to the outflow of aqueous humor 8 through the trabecular outflow pathway 40 may lead to elevated intra-ocular pressure of the eye, which is a widely recognized risk factor for glaucoma. Resistance through the trabecular outflow pathway 40 may increase due a collapsed or malfunctioning Schlemm's canal 18 and trabecular meshwork 12.

Referring to FIG. 5, as an optical system, the eye 1 is represented by an optical model described by idealized centered and rotationally symmetrical surfaces, entrance and exit pupils, and six cardinal points: object and image space focal points, first and second principal planes, and first and second nodal points. Angular directions relative to the human eye are often defined with respect to an optical axis 24, a visual axis 26, a pupillary axis 28 and a line of sight 29 of the eye. The optical axis 24 is the symmetry axis, the line connecting the vertices of the idealized surfaces of the eye. The visual axis 26 connects the foveal center 22 with the first and second nodal points to the object. The line of sight 29 connects the fovea through the exit and entrance pupils to the object. The pupillary axis 28 is normal to the anterior surface of the cornea 3 and directed to the center of the entrance pupil. These axes of the eye differ from one another only by a few degrees and fall within a range of what is generally referred to as the direction of view.

Glaucoma

Glaucoma is a group of diseases that can harm the optic nerve and cause vision loss or blindness. It is the leading cause of irreversible blindness. Approximately 80 million people are estimated to have glaucoma worldwide and of these, approximately 6.7 million are bilaterally blind. More than 2.7 million Americans over age 40 have glaucoma. Symptoms start with loss of peripheral vision and can progress to blindness.

There are two forms of glaucoma, one is referred to as closed-angle glaucoma, the other as open-angled glaucoma. With reference to FIGS. 1-4, in closed-angle glaucoma, the iris 9 in a collapsed anterior chamber 7 may obstruct and close off the flow of aqueous humor 8. In open-angle glaucoma, which is the more common form of glaucoma, the permeability of ocular tissue may be affected by irregularities in the juxtacanalicular tissue 17 and inner wall of Schlemm's canal 18a, blockage of tissue in the irido-corneal angle 13 along the trabecular outflow pathway 40.

As previously stated, elevated intra-ocular pressure (TOP) of the eye, which damages the optic nerve, is a widely recognized risk factor for glaucoma. However, not every person with increased eye pressure will develop glaucoma, and glaucoma can develop without increased eye pressure. Nonetheless, it is desirable to reduce elevated IOP of the eye to reduce the risk of glaucoma.

Methods of diagnosing conditions of the eye of a patient with glaucoma include visual acuity tests and visual field tests, dilated eye exams, tonometry, i.e. measuring the intra-ocular pressure of the eye, and pachymetry, i.e. measuring the thickness of the cornea. Deterioration of vision starts with the narrowing of the visual field and progresses to total blindness. Imaging methods include slit lamp examination, observation of the irido-corneal angle with a gonioscopic lens and optical coherence tomography (OCT) imaging of the anterior chamber and the retina

Once diagnosed, some clinically proven treatments are available to control or lower the intra-ocular pressure of the eye to slow or stop the progress of glaucoma. The most common treatments include: 1) medications, such as eye drops or pills, 2) laser surgery, and 3) traditional surgery. Treatment usually begins with medication. However, the efficacy of medication is often hindered by patient non-compliance. When medication does not work for a patient, laser surgery is typically the next treatment to be tried. Traditional surgery is invasive, more high risk than medication and laser surgery, and has a limited time window of effectiveness. Traditional surgery is thus usually reserved as a last option for patients whose eye pressure cannot be controlled with medication or laser surgery.

Laser Surgery

With reference to FIG. 2, laser surgery for glaucoma targets the trabecular meshwork 12 to decrease aqueous humor 8 flow resistance. Common laser treatments include Argon Laser Trabeculoplasty (ALT), Selective Laser Trabeculoplasty (SLT) and Excimer Laser Trabeculostomy (ELT).

ALT was the first laser trabeculoplasty procedure. During the procedure, an argon laser of 514 nm wavelength is applied to the trabecular meshwork 12 around 180 degrees of the circumference of the irido-corneal angle 13. The argon laser induces a thermal interaction with the ocular tissue that produces openings in the trabecular meshwork 12. ALT, however, causes scarring of the ocular tissue, followed by inflammatory responses and tissue healing that may ultimately close the opening through the trabecular meshwork 12 formed by the ALT treatment, thus reducing the efficacy of the treatment. Furthermore, because of this scarring, ALT therapy is typically not repeatable.

SLT is designed to lower the scarring effect by selectively targeting pigments in the trabecular meshwork 12 and reducing the amount of heat delivered to surrounding ocular tissue. During the procedure, a solid-state laser of 532 nm wavelength is applied to the trabecular meshwork 12 between 180 to 360 degrees around the circumference of the irido-corneal angle 13 to remove the pigmented cells lining the trabeculae which comprise the trabecular meshwork. The collagen ultrastructure of the trabecular meshwork is preserved during SLT. 12. SLT treatment can be repeated, but subsequent treatments have lower effects on TOP reduction.

ELT uses a 308 nm wavelength ultraviolet (UV) excimer laser and non-thermal interaction with ocular tissue to treat the trabecular meshwork 12 and inner wall of Schlemm's canal in a manner that does not invoke a healing response. Therefore, the TOP lowering effect lasts longer. However, because the UV light of the laser cannot penetrate deep into the eye, the laser light is delivered to the trabecular meshwork 12 via an optical fiber inserted into the eye 1 through an opening and the fiber is brought into contact with the trabecular meshwork. The procedure is highly invasive and is generally practiced simultaneously with cataract procedures when the eye is already surgically open. Like ALT and SLT, ELT also lacks control over the amount of TOP reduction.

None of these existing laser treatments represents an ideal treatment for glaucoma. Accordingly, what is needed are systems, apparatuses, and method for laser surgery treatment of glaucoma that effectively reduce TOP non-invasively without significant scarring of tissue, so the treatment may be completed in a single procedure and repeated at a later time if necessary.

Such laser treatment may require the targeting of specific structures of ocular tissue in the irido-corneal angle. For example, during glaucoma surgery with a femtosecond laser, apertures or canals may be created in the trabecular meshwork beginning from the inner wall of Schlemm's canal and extending to the anterior chamber. Focusing the femtosecond laser on the inner wall of the Schlemm's canal is difficult because the focus of the femtosecond laser is not made visible by conventional glaucoma surgical systems. The use of an Optical Coherence Tomography (OCT) imaging system to target the femtosecond laser focus onto the tissue has been suggested. However, the resolution of OCT imaging is limited due to corneal birefringence and dispersion. A high resolution OCT's effective imaging depth is limited to one or two millimeters whereas the width of the anterior chamber, and thus location of the trabecular meshwork, varies by as much as 2 millimeters in the population (11.5-13.5 mm). In addition, the inclusion of an OCT system in the surgical laser increases price and further complicates the surgical laser system. Accordingly, what is further needed in the field of laser treatment of glaucoma are systems and methods that enable visualization and monitoring of a focus of a femtosecond laser and the locating of a focus at or near a target structure of ocular tissue for photodisruption by a femtosecond laser, independent of OCT imaging.

### SUMMARY

The present disclosure relates to a method of locating a structure in an irido-corneal angle of an eye. The structure may be, for example, a surface of the trabecular meshwork, the inner wall of the Schlemm's canal, the outer wall of Schlemm's canal, or the interior of the Schlemm's canal. The method includes directing a laser beam toward the irido-corneal angle of the eye and advancing a focus of the laser beam to a location in the irido-corneal angle that is at or near the structure. During the directing and advancing of the laser, the laser is maintained at a power level insufficient to affect tissue.

The method further includes determining that the focus is at or near the structure based on changes in an intensity of a spot of second harmonic light generated by an encounter between the focus and tissue. To this end, a plurality of intensity states of a spot of second harmonic light are detected, either visually by a surgeon viewing the spot on a display or automatically by a processor analyzing the output of a photodetector or analyzing captured images of the spot, as the focus of the laser is advanced. Each of the plurality of intensity states corresponds to one of a presence of the spot of second harmonic light or an absence of the spot of second harmonic light. The plurality of intensity states defines a pattern that maps to the structure. Thus, upon detection of the pattern, it may be concluded that the focus is at or near the structure.

In one example, the structure is a proximal surface of a trabecular meshwork and the detected pattern corresponds to an absence of the spot of second harmonic light followed by a presence of the spot of second harmonic light. In another example, the structure is an inner wall of a Schlemm's canal and the detected pattern corresponds to an absence of the spot of second harmonic light, followed by a presence of the spot of second harmonic light, followed by an absence of the spot of second harmonic light. In another example, the structure is an outer wall of a Schlemm's canal and the detected pattern corresponds to an absence of the spot of second harmonic light, followed by a presence of the spot of second harmonic light, followed by an absence of the spot of second harmonic light, followed by a presence of the spot of second harmonic light. In yet another example, the structure is an interior of a Schlemm's canal and the detected pattern corresponds to an absence of the spot of second harmonic light, followed by a presence of the spot of second harmonic light, followed by an absence of the spot of second harmonic light.

Considering the method further, in an additional aspect, laser treatment of the structure by the laser at the focus is initiated after the focus is determined to be at or near the structure. Initiating laser treatment comprises increasing a power level of the laser to a level sufficient to photodisrupt tissue. A layer or plane of tissue is then photodisrupted by scanning the focus.

The present disclosure also relates to a system for locating a structure in an irido-corneal angle of an eye. The system includes a focusing objective configured to be coupled to the eye. The focusing objective is aligned to receive a laser beam and to direct the laser beam toward the irido-corneal angle of the eye. The system also includes a laser subsystem configured to output the laser beam to the focusing objective, and a second harmonic light detection apparatus configured to detect a spot of second harmonic light generated by an encounter between a focus of the laser and tissue.

A control system coupled to the laser subsystem and the second harmonic light detection apparatus is configured to advance the focus of the laser beam to a location in the irido-corneal angle that is at or near the structure. The control system is further configured to determine that the focus is at or near the structure based on changes in an intensity of the spot of second harmonic light. To this end, the control system is configured to detect a plurality of intensity states of the spot of second harmonic light as the focus of the laser is advanced. Each of the plurality of intensity states corresponds to one of a presence of the spot of second harmonic light or an absence of the spot of second harmonic light. The plurality of intensity states defines a pattern that maps to the structure. Thus, upon detection of the pattern, the control system concludes that the focus is at or near the structure.

It is understood that other aspects of apparatuses and methods will become apparent to those skilled in the art from the following detailed description, wherein various aspects of apparatuses and methods are shown and described by way of illustration. As will be realized, these aspects may be implemented in other and different forms and its several details are capable of modification in various other respects. Accordingly, the drawings and detailed description are to be regarded as illustrative in nature and not as restrictive.

### BRIEF DESCRIPTION OF THE DRAWINGS

Various aspects of systems and methods will now be presented in the detailed description by way of example, and not by way of limitation, with reference to the accompanying drawings, wherein:

FIG. 1 is a sectional schematic illustration of a human eye and its interior anatomical structures.

FIG. 2 is a sectional schematic illustration of the irido-corneal angle of the eye of FIG. 1.

FIG. 3 is a sectional schematic illustration detailing anatomical structures in the irido-corneal angle of FIG. 2, including the trabecular meshwork, Schlemm's canal, and one or more collector channels branching from the Schlemm's canal.

FIG. 4 is a sectional schematic illustration of various outflow pathways for aqueous humor through the trabecular meshwork, Schlemm's canal, and collector channels of FIG. 3.

FIG. 5 is a sectional schematic illustration of a human eye showing various axes associated with the eye.

FIG. 6 is a sectional schematic illustration of an angled beam path along which one or more light beams may access the irido-corneal angle of the eye.

FIG. 7 is a block diagram of an integrated surgical system for non-invasive glaucoma surgery including a control system, a laser source, an OCT imaging apparatus, a visual observation apparatus, a second harmonic light detection apparatus, beam conditioners and scanners, beam combiners, a focusing objective, and a patient interface.

FIG. 8 is a detailed block diagram of the integrated surgical system of FIG. 7.

FIGS. 9a and 9b are schematic illustrations of the focusing objective of the integrated surgical system of FIG. 7 coupled to (FIG. 9a) and decoupled from (FIG. 9b) the patient interface of the integrated surgical system of FIG. 7.

FIG. 9c is a schematic illustration of components of the focusing objective and the patient interface included in FIGS. 9a and 9b.

FIGS. 10a and 10b are schematic illustrations of components of the integrated surgical system of FIGS. 7 and 8 functionally arranged to form a first optical system and a second optical subsystem that enable access to the to the irido-corneal angle along the angled beam path of FIG. 6.

FIG. 10c is a schematic illustration of a beam passing through the first optical subsystem of FIGS. 10a and 10b and into the eye.

FIG. 11 is a three-dimensional schematic illustration of anatomical structures in the irido-corneal angle, including the trabecular meshwork, Schlemm's canal, a collector channel branching from the Schlemm's canal, and a surgical volume of ocular tissue to be treated by the integrated surgical system of FIG. 7.

FIG. 12 is a two-dimensional schematic illustration of anatomical structures in the irido-corneal angle and a laser treatment pattern to be applied by the integrated surgical system of FIG. 7 to affect the surgical volume of ocular tissue between the Schlemm's canal and the anterior chamber, as shown in FIG. 11.

FIG. 13 is a three-dimensional schematic illustration of FIG. 11 subsequent to treatment of the surgical volume of ocular tissue by a laser based on the laser treatment pattern of FIG. 12 that forms an opening between the Schlemm's canal and the anterior chamber.

FIGS. 14a-14g are a series of a schematic illustrations of a focus of a femtosecond laser and corresponding spots of second harmonic light, if any, as the focus is advanced from the anterior chamber (FIG. 14a), through the trabecular meshwork (FIGS. 14b and 14c), through the inner wall of the Schlemm's canal into the canal (FIG. 14d), through the interior of the Schlemm's canal to the outer wall of the canal (FIG. 14e), and then back to the inner wall of the Schlemm's canal (FIG. 14f) or the interior of the Schlemm's canal (FIG. 14g).

FIG. 15 is a schematic illustration of a laser scanning process that produces the opening between the Schlemm's canal and the anterior chamber, as shown in FIG. 13, where the scanning begins at the inner wall of the Schlemm's canal and proceeds toward the anterior chamber.

FIG. 16 is a flowchart of a method of locating a target structure of ocular tissue in an irido-corneal angle of an eye for photodisruption by a femtosecond laser.

FIG. 17 is an example profile of the intensity of second harmonic generated (SHG) light as a function of laser focus depth detected from a human cadaver eye.

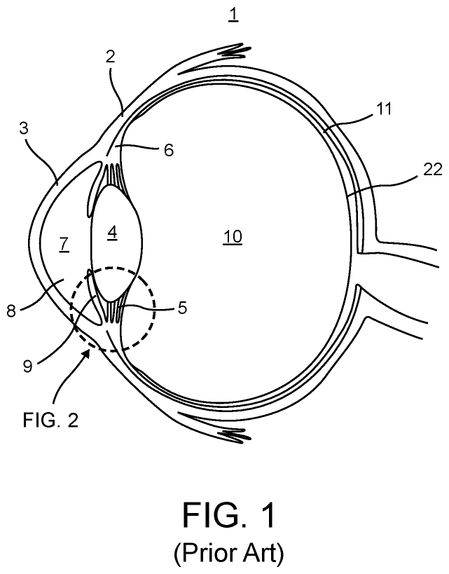

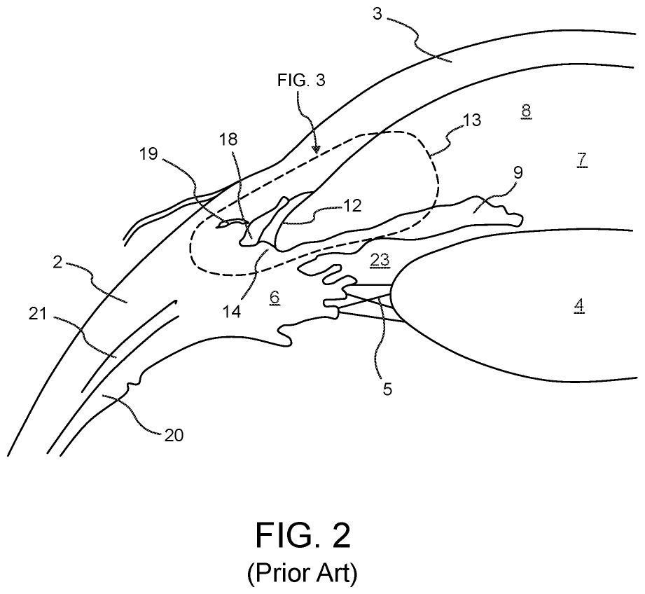

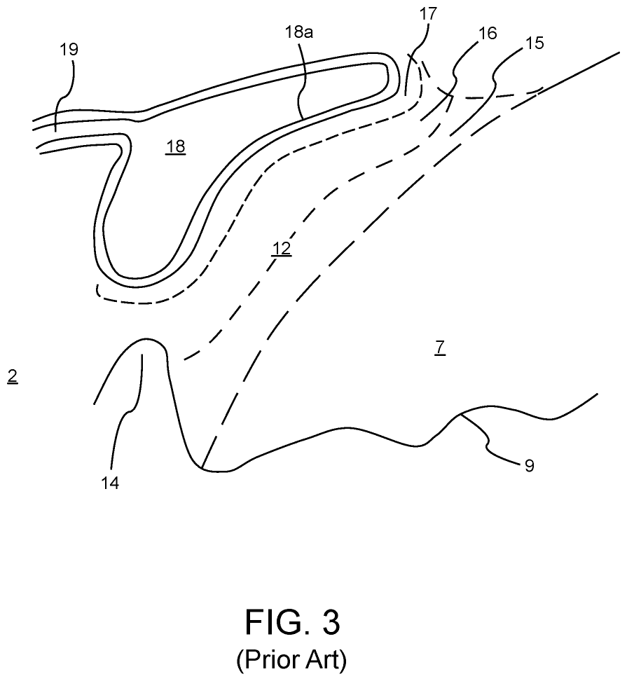

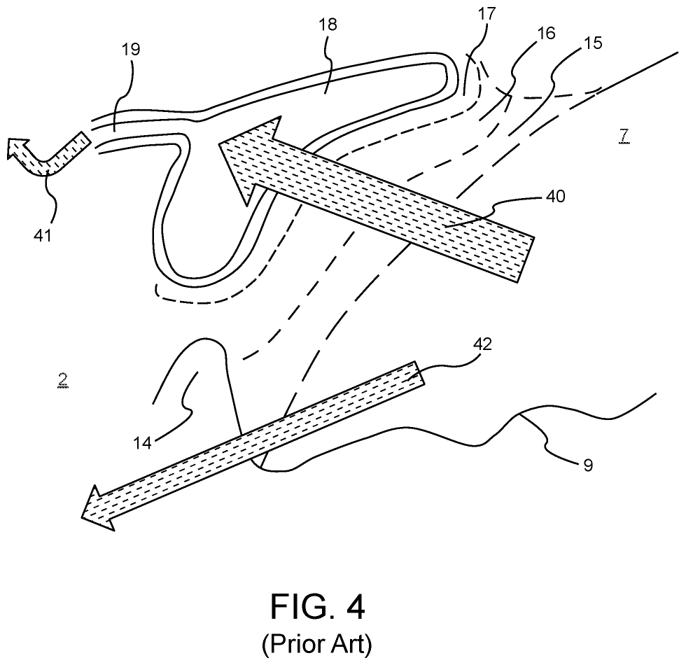

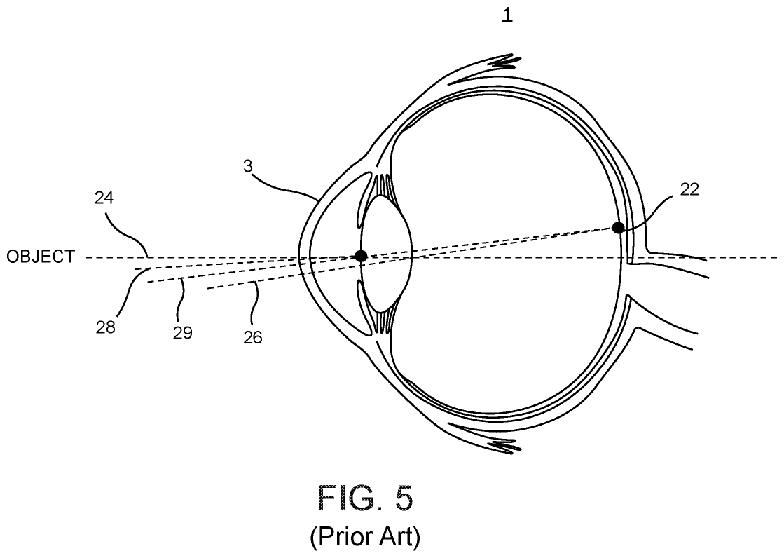

### DETAILED DESCRIPTION

Disclosed herein are systems and methods for safely and effectively reducing intra-ocular pressure (TOP) in the eye to either treat or reduce the risk of glaucoma. The systems and methods enable access to the irido-corneal angle of the eye and use laser surgery techniques to treat abnormal ocular tissue conditions within the irido-corneal angle that may be causing elevated IOP. The system and method also enable the locating of a focus of a femtosecond laser at or near a target structure of ocular tissue for laser surgery.

A system disclosed herein locates a target structure in an irido-corneal angle of an eye. The system includes a focusing objective configured to be coupled to the eye. The focusing objective is aligned to receive a laser beam and to direct the laser beam toward the irido-corneal angle of the eye. The system also includes a laser subsystem configured to output the laser beam, and a second harmonic light detection apparatus configured to detect a spot of second harmonic light generated by an encounter between the focus and tissue. The laser is configured to produce optical pulses of sufficiently short duration, e.g., 1 nanosecond or less. These short duration pulses enable the generation of second harmonic light. They also enable the photodisruption of ocular tissue, provided the power level of the pulses is at a sufficient level. In one embodiment, the laser is a femtosecond laser. In another embodiment, the laser is a Q-switched Nd:YAG laser.

A control system is coupled to the laser subsystem and the second harmonic light detection apparatus. The control system is configured to advance a focus of the femtosecond laser beam to a location in the irido-corneal angle that is at or near the target structure, and to determine that the focus is at or near the target structure based on changes in an intensity of the spot of second harmonic light. To this end, the control system is configured to detect a plurality of intensity states of a spot of second harmonic light as the focus of the femtosecond laser is advanced. Each of the plurality of intensity states corresponds to one of a presence of the spot of second harmonic light or an absence of the spot of second harmonic light. The plurality of intensity states define a pattern that maps to the target structure. Thus, upon detection of the pattern, the control system concludes that the focus is at or near the target structure.

The system disclosed herein enables the detection of a focus of a laser, operating at low power, as the focus is being advanced toward a target structure. The detection may be made either visually by a surgeon viewing the irido-corneal angle and observing a spot of second harmonic light corresponding to the focus on a display, or automatically by a processor analyzing the output of a photodetector that is directed at the irido-corneal angle and configured to detect second harmonic light, or analyzing video images of the irido-corneal angle captured by a camera configured to detect second harmonic light. In either case, the detection is based on a spot of second harmonic light generated by an encounter between the focus and tissue, which is captured by the second harmonic light detection apparatus, independent of additional instrumentation, such as an OCT imaging apparatus.

Second Harmonic Light Detection

A second harmonic light detection apparatus is used by the system disclosed herein to detect spots of second harmonic light generated by an encounter between a focus of a femtosecond laser and a tissue surface. Such generation from a surface encounter is referred to as a surface second harmonic generation. This second harmonic generation is a nonlinear optical process that effectively combines two photons of frequency ω to generate a single photon of frequency 2w. Materials that exhibit second harmonic generation are characterized by a second order non-linear susceptibility, χ(2)>0, i.e., and do not possess inversion symmetry. In the case of a surface, inversion symmetry is naturally broken by the surface itself. Therefore if the number of photons in a given area is large (as in a tightly focused beam) then second harmonic generation is generated from the interaction volume.

The second harmonic light detection apparatus is configured to optically couple with a focusing objective to establish a line of sight with the irido-corneal angle of an eye and to detect spots of second harmonic light generated by laser interaction with ocular tissue in the irido-corneal angle. In one configuration, the second harmonic light detection apparatus is a photodetector configured to optically couple with a focusing objective to establish a line of sight with the irido-corneal angle of an eye to photodetect spots of second harmonic light. Such a photodetector may be, for example, instrumented with a visible filter centered near or at 515 nm (with a bandpass of 20-50 nm) that detects green light generated by surface second harmonic generation.

In another configuration, the second harmonic light detection apparatus is a video camera that enables the detection of spots of second harmonic light through video imaging of the irido-corneal angle. The video camera may be a standard ophthalmic video camera, or a standard ophthalmic video camera instrumented with a visible filter centered near or at 515 nm (with a bandpass of 20-50 nm) that detects green light generated by surface second harmonic generation. Spots of second harmonic light may be detected, either through observation of the video image on a display by the surgeon, or automatically through image processing of the video image.

In either configuration, as a focus of a femtosecond laser is advanced through the anterior chamber of the eye and within the irido-corneal angle, the focus encounters areas that are void of tissue surfaces and areas where a tissue surface is present. Areas that are void of tissue surfaces do not result in second harmonic generation and thus a spot of second harmonic light is not detected by the second harmonic light detection apparatus. Areas with a tissue surface do result in second harmonic generation and thus a spot of second harmonic light is detected.

OCT Imaging

While the system disclosed herein enables the locating of a target structure of ocular tissue in an irido-corneal angle of an eye for photodisruption by a femtosecond laser without the use of OCT imaging, OCT imaging may be used by the system to provide spatial resolution and contrast to resolve microscopic details of ocular tissue. When used, OCT imaging can provide two-dimensional (2D) cross-sectional images of the ocular tissue. These 2D cross-sectional images may be processed and analyzed to determine the size, shape and location of structures in the eye.

Femtosecond Laser Source

The preferred laser surgical component of the integrated surgical system disclosed herein is a femtosecond laser. A femtosecond laser provides highly localized, non-thermal photo-disruptive laser-tissue interaction with minimal collateral damage to surrounding ocular tissue. Photo-disruptive interaction of the laser is utilized in optically transparent tissue. The principal mechanism of laser energy deposition into the ocular tissue is not by absorption but by a highly nonlinear multiphoton process. This process is effective only at the focus of the pulsed laser where the peak intensity is high. Regions where the beam is traversed but not at the focus are not affected by the laser. Therefore, the interaction region with the ocular tissue is highly localized both transversally and axially along the laser beam. The process can also be used in weakly absorbing or weakly scattering tissue. While femtosecond lasers with photo-disruptive interactions have been successfully used in ophthalmic surgical systems and commercialized in other ophthalmic laser procedures, none have been used in an integrated surgical system that accesses the irido-corneal angle.

In known refractive procedures, femtosecond lasers are used to create corneal flaps, pockets, tunnels, arcuate incisions, lenticule shaped incisions, partial or fully penetrating corneal incisions for keratoplasty. For cataract procedures the laser creates a circular cut on the capsular bag of the eye for capsulotomy and incisions of various patterns in the lens for breaking up the interior of the crystalline lens to smaller fragments to facilitate extraction. Entry incisions through the cornea opens the eye for access with manual surgical devices and for insertions of phacoemulsification devices and intra-ocular lens insertion devices.

These existing systems are developed for their specific applications, for surgery in the cornea, and the crystalline lens and its capsular bag and are not capable of performing surgery in the irido-corneal angle 13 for several reasons. First, the irido-corneal angle 13 is not accessible with these surgical laser systems because the irido-corneal angle is too far out in the periphery and is outside of surgical range of these systems. Second, the angle of the laser beam from these systems, which is along the optical axis 24 to the eye 1, is not appropriate to reaching the irido-corneal angle 13, where there is significant scattering and optical distortion at the applied wavelength. Third, any imaging capabilities these systems may have do not have the accessibility, penetration depth and resolution to image the tissue along the trabecular outflow pathway 40 with sufficient detail and contrast.

In the integrated surgical system disclosed herein, clear access to the irido-corneal angle 13 is provided along the angled beam path 30. The tissue, e.g., cornea 3 and the aqueous humor 8 in the anterior chamber 7, along this angled beam path 30 is transparent for wavelengths from approximately 400 nm to 2500 nm and femtosecond lasers operating in this region can be used. Such mode locked lasers work at their fundamental wavelength with Titanium, Neodymium or Ytterbium active material. Non-linear frequency conversion techniques known in the art, frequency doubling, tripling, sum and difference frequency mixing techniques, optical parametric conversion can convert the fundamental wavelength of these lasers to practically any wavelength in the above mentioned transparent wavelength range of the cornea.

Existing ophthalmic surgical systems apply lasers with pulse durations longer than 1 ns have higher photo-disruption threshold energy, require higher pulse energy and the dimension of the photo-disruptive interaction region is larger, resulting in loss of precision of the surgical treatment. When treating the irido-corneal angle 13, however, higher surgical precision is required. To this end, the integrated surgical system may be configured to apply lasers with pulse durations from 10 femtosecond (fs) to 1 nanosecond (ns) for generating photo-disruptive interaction of the laser beam with ocular tissue in the irido-corneal angle 13. While lasers with pulse durations shorter than 10 fs are available, such laser sources are more complex and more expensive. Lasers with the described desirable characteristics, e.g., pulse durations from 10 femtosecond (fs) to 1 nanosecond (ns), are commercially available.

Accessing the Irido-corneal Angle

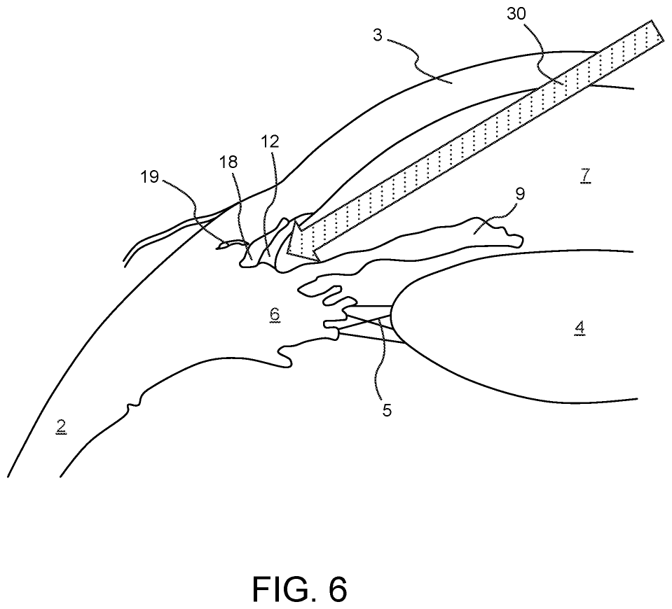

An important feature afforded by the integrated surgical system is access to the targeted ocular tissue in the irido-corneal angle 13. With reference to FIG. 6, the irido-corneal angle 13 of the eye may be accessed via the integrated surgical system along an angled beam path 30 passing through the cornea 3 and through the aqueous humor 8 in the anterior chamber 7. For example, one or more of an imaging beam, e.g., an OCT beam and/or a visual observation beam, and a laser beam may access the irido-corneal angle 13 of the eye along the angled beam path 30.

An optical system disclosed herein is configured to direct a light beam to an irido-corneal angle 13 of an eye along an angled beam path 30. The optical system includes a first optical subsystem and a second optical subsystem. The first optical subsystem includes a window formed of a material with a refractive index n_{w} and has opposed concave and convex surfaces. The first optical subsystem also includes an exit lens formed of a material having a refractive index nₓ. The exit lens also has opposed concave and convex surfaces. The concave surface of the exit lens is configured to couple to the convex surface of the window to define a first optical axis extending through the window and the exit lens. The concave surface of the window is configured to detachably couple to a cornea of the eye with a refractive index n, such that, when coupled to the eye, the first optical axis is generally aligned with the direction of view of the eye.

The second optical subsystem is configured to output a light beam, e.g., an OCT beam or a laser beam. The optical system is configured so that the light beam is directed to be incident at the convex surface of the exit lens along a second optical axis at an angle α that is offset from the first optical axis. The respective geometries and respective refractive indices nₓ, and n_{w} of the exit lens and window are configured to compensate for refraction and distortion of the light beam by bending the light beam so that it is directed through the cornea 3 of the eye toward the irido-corneal angle 13. More specifically, the first optical system bends the light beam such that the light beam exits the first optical subsystem and enters the cornea 3 at an appropriate angle so that the light beam progresses through the cornea and the aqueous humor 8 in a direction along the angled beam path 30 toward the irido-corneal angle 13.

Accessing the irido-corneal angle 13 along the angled beam path 30 provides several advantages. An advantage of this angled beam path 30 to the irido-corneal angle 13 is that the OCT beam and laser beam passes through mostly clear tissue, e.g., the cornea 3 and the aqueous humor 8 in the anterior chamber 7. Thus, scattering of these beams by tissue is not significant. With respect to OCT imaging, this enables the use of shorter wavelength, less than approximately 1 micrometer, for the OCT to achieve higher spatial resolution. An additional advantage of the angled beam path 30 to the irido-corneal angle 13 through the cornea 3 and the anterior chamber 7 is the avoidance of direct laser beam or OCT beam light illuminating the retina 11. As a result, higher average power laser light and OCT light can be used for imaging and surgery, resulting in faster procedures and less tissue movement during the procedure.

Another important feature provided by the integrated surgical system is access to the targeted ocular tissue in the irido-corneal angle 13 in a way that reduces beam discontinuity. To this end, the window and exit lens components of the first optical subsystem are configured to reduce the discontinuity of the optical refractive index between the cornea 3 and the neighboring material and facilitate entering light through the cornea at a steep angle.

Having thus generally described the integrated surgical system and some of its features and advantages, a more detailed description of the system and its component parts follows.

Integrated Surgical System

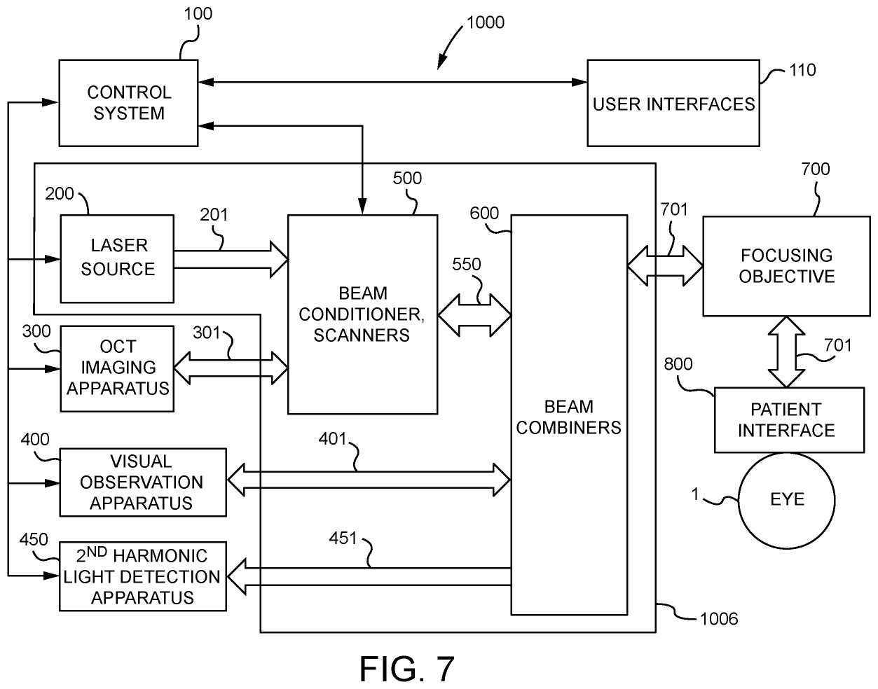

With reference to FIG. 7, an integrated surgical system 1000 for non-invasive glaucoma surgery includes a control system 100, a surgical component 200, a first imaging apparatus 300 and a second imaging apparatus 400, and a second harmonic light detection apparatus 450. In the embodiment of FIG. 7, the surgical component 200 is a femtosecond laser source, the first imaging apparatus 300 is an OCT imaging apparatus, and the second imaging apparatus 400 is a visual observation apparatus comprising a video camera and an illumination source for viewing or capturing images of a surgical field. The second harmonic light detection apparatus 450 may be, for example, a photodetector configured to detect second harmonic light generated in the surgical field or a video camera instrumented with a visible filter centered near or at 515 nm (with a bandpass of 20-50 nm) that detects green light generated by surface second harmonic generation. Other components of the integrated surgical system 1000 include beam conditioners and scanners 500, beam combiners 600, a focusing objective 700, and a patient interface 800.

The control system 100 may be a single computer or and plurality of interconnected computers configured to control the hardware and software components of the other components of the integrated surgical system 1000. A user interface 110 of the control system 100 accepts instructions from a user and displays information for observation by the user. Input information and commands from the user include but are not limited to system commands, motion controls for docking the patient's eye to the system, selection of pre-programmed or live generated surgical plans, navigating through menu choices, setting of surgical parameters, responses to system messages, determining and acceptance of surgical plans and commands to execute the surgical plan. Outputs from the system towards the user includes but are not limited to display of system parameters and messages, display of images of the eye, graphical, numerical and textual display of the surgical plan and the progress of the surgery.

The control system 100 is connected to the other components 200, 300, 400, 450, 500 of the integrated surgical system 1000. Signals between the control system 100 and the femtosecond laser source 200 function to control internal and external operation parameters of the laser source, including for example, power, repetition rate and beam shutter. Signals between the control system 100 and the OCT imaging apparatus 300 function to control OCT beam scanning parameters, and the acquiring, analyzing and displaying of OCT images. Signals between the control system 100 and the second harmonic light detection apparatus 450 function to control the operation of the second harmonic light detection apparatus, and the detecting of second harmonic light generated by an encounter between the focus of the laser and tissue in the irido-corneal angle of the eye. To this end, the line of sight of the second harmonic light detection apparatus 450 is aligned with the femtosecond laser and directed into the irido-corneal angle of the eye. Signals between the control system 100 and the visual observation apparatus 400 function to control the capturing and displaying of images of the surgical field. To this end, the line of sight of the visual observation apparatus 400 is aligned with the femtosecond laser and directed into the irido-corneal angle of the eye. Control signals from the control system 1000 to the beam conditioner and scanners 500 function to control the focus of the laser beam output by the femtosecond laser source 200. Such control may include advancing the focus of the laser beam in the direction of propagation of the laser or in the direction opposite the direction of propagation of the laser, and scanning the focus.

Laser beams 201 from the femtosecond laser source 200 and OCT beams 301 from the OCT imaging apparatus 300 are directed towards a unit of beam conditioners and scanners 500. Different kinds of scanners can be used for the purpose of scanning the laser beam 201 and the OCT beam 301. For scanning transversal to a beam 201, 301, angular scanning galvanometer scanners are available for example from Cambridge Technology, Bedford, Mass., and Scanlab, Munich, Germany. To optimize scanning speed, the scanner mirrors are typically sized to the smallest size, which still support the required scanning angles and numerical apertures of the beams at the target locations. The ideal beam size at the scanners is typically different from the beam size of the laser beam 201 or the OCT beam 301, and different from what is needed at the entrance of a focusing objective 700. Therefore, beam conditioners are applied before, after or in between individual scanners. The beam conditioner and scanners 500 includes scanners for scanning the beam transversally and axially. Axial scanning changes the depth of the focus at the target region. Axial scanning can be performed by moving a lens axially in the beam path with a servo or stepper motor.

The laser beam 201 and the OCT beam 301 are combined by dichroic, polarization or other kind of beam combiners 600 to reach a common target volume or surgical volume in the eye. Likewise, an illumination beam 401 from the visual observation apparatus 400 may be combined with the laser beam 201 and the OCT beam 301 by dichroic, polarization or other kind of beam combiners 600 to reach the common target volume or surgical volume in the eye. In an integrated surgical system 1000 having a femtosecond laser source 200, an OCT imaging apparatus 300, and a visual observation apparatus 400, the individual beams 201, 301, 401 for each of these components may be individually optimized and may be collinear or non-collinear to one another. The beam combiner 600 uses dichroic or polarization beam splitters to split and recombine light with different wavelength and/or polarization. The beam combiner 600 may also include optics, such as a telescope, to change certain parameters of the individual beams 201, 301, 401 such as beam size, beam angle and divergence. Integrated visual illumination, observation or imaging devices assist the surgeon in docking the eye to the system and identifying surgical locations.

To facilitate locating a focus of a femtosecond laser beam 201 at or near a target structure of ocular tissue, the second harmonic light detection apparatus 450 of the integrated surgical system 1000 generates information indicative of the presence or absence of second harmonic light in the irido-corneal angle of the eye. To this end, in one embodiment, the second harmonic light detection apparatus 450 is configured to detect for a second harmonic light beam 451 using a photodetector, and to provide an intensity profile of second harmonic generated light as a function of scan depth of the second harmonic signal as the focus of the femtosecond laser beam 201 is advanced.

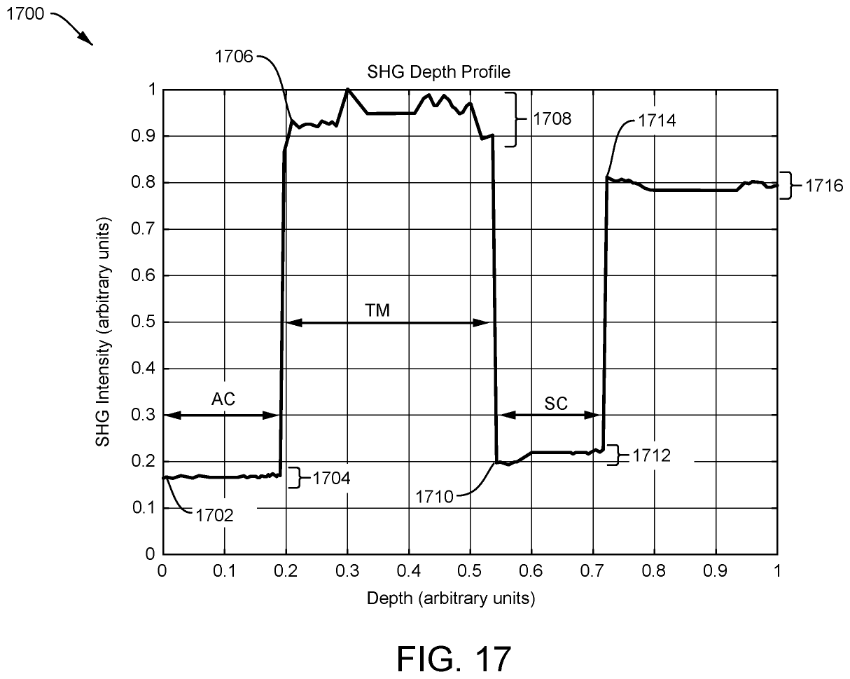

With reference to FIG. 17, an example intensity profile 1700 of the average intensity of second harmonic generated (SHG) light as a function of laser focus depth detected by a second harmonic light detection apparatus 450 from a human cadaver eye is shown. In the SHG intensity profile 1700, the level or measure of SHG light intensity (vertical axis) and depth (horizontal axis) in the direction of propagation of the laser are in arbitrary units scaled from 0 to 1. In practice, the actual values of units, which may vary depending on the setup of the surgical system and electrical offsets that require calibration, as not particularly relevant in that the most significant information is gleaned from the relative changes in SHG light intensity exhibited in the SHG intensity profile 1700.

Continuing with FIG. 17, the intensity of SHG light starts at a nominal low level 1702 within a first low-level range 1704 while the focus of the femtosecond laser is inside the anterior chamber (AC). The SHG intensity remains within the first low-level range 1704 until the focus of the femtosecond laser encounters the proximal surface or inner wall of the trabecular meshwork (TM). At the inner wall of the trabecular meshwork, the SHG intensity increase to a high level 1706 within a first high-level range 1708 and remains within the first high-level range until the focus of the femtosecond laser enters Schlemm's canal (SC), at which time the SHG intensity drops to a low level 1710 within a second low-level range 1712. Notice that the SHG intensity did not drop back to the original nominal level 1702 when the focus entered the Schlemm's canal. Instead the SHG intensity level inside the Schlemm's canal is within the second low-level range 1712, which is slightly higher than the first low-level range 1704 within the AC. Presumedly a small amount of SHG light is created by red blood cells or other material within Schlemm's canal, thus resulting in a slightly higher intensity relative to the AC. Finally, as the focus of the femtosecond laser encounters the outer wall of the Schlemm's canal, the SHG intensity increase to a high level 1714 within a second high-level range 1716. Like the first and second low-level ranges 1407, 1712 the second high-level range 1716 is slightly less than the first high-level range 1708 associated with the trabecular meshwork (TM). Based on the varying SHG intensity levels provided by the second harmonic light detection apparatus 450, the control system 100 is able to monitor the location of the focus of the femtosecond laser and determine the tissue structure, e.g., anterior chamber, trabecular meshwork, Schlemm's canal, at which the focus is located.

The second harmonic signal is at its maximum when the tissue surface being encountered by the femtosecond laser beam is at the beam waste of the laser beam. Accordingly, a maximum of the second harmonic signal indicates that the tissue surface is at the focal point of the laser beam. When a focus of the femtosecond laser is determined to be at a tissue surface, the depth location of that tissue surface may be determined from the position of the focusing objective 700 and the focal length of the focusing objective.

To resolve ocular tissue structures of the eye in sufficient detail, the OCT imaging apparatus 300 of the integrated surgical system 1000 may provide an OCT having a spatial resolution of several micrometers. The resolution of the OCT beam is the spatial dimension of the smallest feature that can be recognized in the OCT image. It is determined mostly by the wavelength and the spectral bandwidth of the OCT source, the quality of the optics delivering the OCT beam to the target location in the eye, the numerical aperture of the OCT beam and the spatial resolution of the OCT imaging apparatus 300 at the target location. In one embodiment, the OCT beam of the integrated surgical system has a resolution of no more than 5 μm.

Likewise, the surgical laser beam provided by the femtosecond laser source 200 may be delivered to targeted locations with several micrometer accuracy. The resolution of the laser beam is the spatial dimension of the smallest feature at the target location that can be modified by the laser beam without significantly affecting surrounding ocular tissue. It is determined mostly by the wavelength of the laser beam, the quality of the optics delivering the laser beam to target location in the eye, the numerical aperture of the laser beam, the energy of the laser pulses in the laser beam and the spatial resolution of the laser scanning system at the target location. In addition, to minimize the threshold energy of the laser for photo-disruptive interaction, the size of the laser spot should be no more than approximately 5 μm.

For practical embodiments, beam conditioning, scanning and the combining of the optical paths are performed on the laser beam 201, OCT beam 301, and the illumination beam 401. Implementation of those functions may happen in a different order than what is indicated in FIG. 7. Specific optical hardware that manipulates the beams to implement those functions can have multiple arrangements with regards to how the optical hardware is arranged. They can be arranged in a way that manipulates individual optical beams separately, in another embodiment one component may combine functions and manipulates different beams. For example, a single set of scanners can scan both the laser beam 201 and the OCT beam 301. In this case, separate beam conditioners set the beam parameters for the laser beam 201 and the OCT beam 301, then a beam combiner combines the two beams for a single set of scanners to scan the beams. While many combinations of optical hardware arrangements are possible for the integrated surgical system, the following section describes an example arrangement.

In the following description, the term beam may—depending on the context—refer to one of a laser beam, an OCT beam, or an illumination beam. A combined beam refers to two or more of a laser beam, an OCT beam, or an illumination beam that are either collinearly combined or non-collinearly combined. Example combined beams include a combined OCT/laser beam, which is a collinear or non-colinear combination of an OCT beam and a laser beam, and a combined OCT/laser/illumination beam, which is a collinear or non-collinear combination of an OCT beam, a laser beam, and an illumination beam. In a collinearly combined beam, the different beams may be combined by dichroic or polarization beam splitters, and delivered along a same optical path through a multiplexed delivery of the different beams. In a non-collinear combined beam, the different beams are delivered at the same time along different optical paths that are separated spatially or by an angle between them. In the description to follow, any of the foregoing beams or combined beams may be generically referred to as a light beam. The terms distal and proximal may be used to designate the direction of travel of a beam, or the physical location of components relative to each other within the integrated surgical system. The distal direction refers to a direction toward the eye; thus an OCT beam output by the OCT imaging apparatus moves in the distal direction toward the eye. The proximal direction refers to a direction away from the eye; thus an OCT return beam from the eye moves in the proximal direction toward the OCT imaging apparatus.

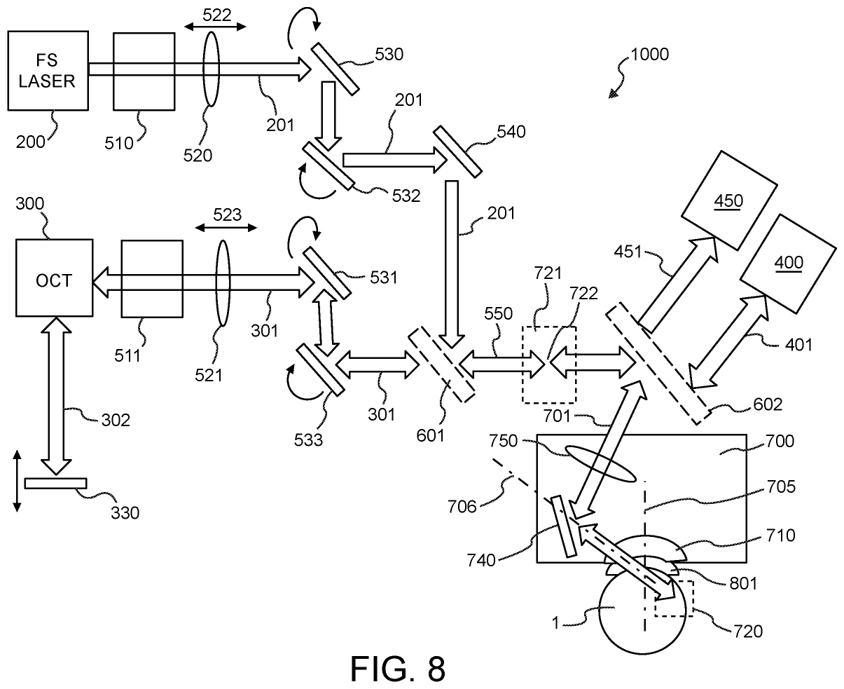

Referring to FIG. 8, in one embodiment, an integrated surgical system is configured to deliver each of a laser beam 201 and an illumination beam 401 in the distal direction toward an eye 1, and receive a visual observation beam 401 and a second harmonic light beam 451 back from the eye 1. In another embodiment, an integrated surgical system is configured to deliver each of a laser beam 201, an OCT beam 301, and an illumination beam 401 in the distal direction toward an eye, and receive each of an OCT return beam 301, a visual observation beam 401, and a second harmonic light beam 451 back from the eye. Regarding the delivery of a laser beam, a laser beam 201 output by the femtosecond laser source 200 passes through a beam conditioner 510 where the basic beam parameters, beam size, divergence are set. The beam conditioner 510 may also include additional functions, setting the beam power or pulse energy and shutter the beam to turn it on or off. After existing the beam conditioner 510, the laser beam 210 enters an axial scanning lens 520. The axial scanning lens 520, which may include a single lens or a group of lenses, is movable in the axial direction 522 by a servo motor, stepper motor or other control mechanism. Movement of the axial scanning lens 520 in the axial direction 522 changes the axial distance of the focus of the laser beam 210 at a focal point.

In a particular embodiment of the integrated surgical system, an intermediate focal point 722 is set to fall within, and is scannable in, the conjugate surgical volume 721, which is an image conjugate of the surgical volume 720, determined by the focusing objective 700. The surgical volume 720 is the spatial extent of the region of interest within the eye where imaging and surgery is performed. For glaucoma surgery, the surgical volume 720 is the vicinity of the irido-corneal angle 13 of the eye.

A pair of transverse scanning mirrors 530, 532 rotated by a galvanometer scanner scan the laser beam 201 in two essentially orthogonal transversal directions, e.g., in the x and y directions. Then the laser beam 201 is directed towards a dichroic or polarization beam splitter 540 where it is reflected toward a beam combining mirror 601 configured to combine the laser beam 201 with an OCT beam 301.

Regarding delivery of an OCT beam, an OCT beam 301 output by the OCT imaging apparatus 300 passes through a beam conditioner 511, an axially moveable focusing lens 521 and a transversal scanner with scanning mirrors 531 and 533. The focusing lens 521 is used to set the focal position of the OCT beam in the conjugate surgical volume 721 and the real surgical volume 720. The focusing lens 521 is not scanned for obtaining an OCT axial scan. Axial spatial information of the OCT image is obtained by Fourier transforming the spectrum of the interferometrically recombined OCT return beam 301 and reference beams 302. However, the focusing lens 521 can be used to re-adjust the focus when the surgical volume 720 is divided into several axial segments. This way the optimal imaging spatial resolution of the OCT image can be extended beyond the Rayleigh range of the OCT signal beam, at the expense of time spent on scanning at multiple ranges.

Proceeding in the distal direction toward the eye 1, after the scanning mirrors 531 and 533, the OCT beam 301 is combined with the laser beam 201 by the beam combiner mirror 601. The OCT beam 301 and laser beam 201 components of the combined laser/OCT beam 550 are multiplexed and travel in the same direction to be focused at an intermediate focal point 722 within the conjugate surgical volume 721. After having been focused in the conjugate surgical volume 721, the combined laser/OCT beam 550 propagates to a second beam combining mirror 602 where it is combined with an illumination beam 401 to form a combined laser/OCT/illumination beam 701.

The combined laser/OCT/illumination beam 701 traveling in the distal direction then passes through an objective lens 750 included in the focusing objective 700, is reflected by a beam-folding mirror 740 and then passes through an exit lens 710 and a window 801 of a patient interface, where the intermediate focal point 722 of the laser beam within the conjugate surgical volume 721 is re-imaged into a focal point in the surgical volume 720. The focusing objective 700 re-images the intermediate focal point 722, through the window 801 of a patient interface, into the ocular tissue within the surgical volume 720.

A scattered OCT return beam 301 from the ocular tissue travels in the proximal direction to return to the OCT imaging apparatus 300 along the same paths just described, in reverse order. The reference beam 302 of the OCT imaging apparatus 300, passes through a reference delay optical path and return to the OCT imaging apparatus from a moveable mirror 330. The reference beam 302 is combined interferometrically with the OCT return beam 301 on its return within the OCT imaging apparatus 300. The amount of delay in the reference delay optical path is adjustable by moving the moveable mirror 330 to equalize the optical paths of the OCT return beam 301 and the reference beam 302. For best axial OCT resolution, the OCT return beam 301 and the reference beam 302 are also dispersion compensated to equalize the group velocity dispersion within the two arms of the OCT interferometer.

When the combined laser/OCT/illumination beam 701 is delivered through the cornea 3 and the anterior chamber 7, the combined beam passes through posterior and anterior surface of the cornea at a steep angle, far from normal incidence. These surfaces in the path of the combined laser/OCT/illumination beam 701 create excessive astigmatism and coma aberrations that need to be compensated for.

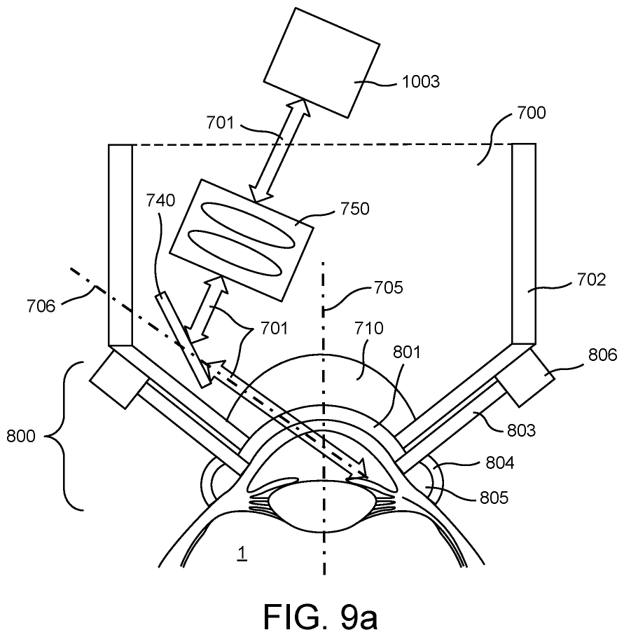

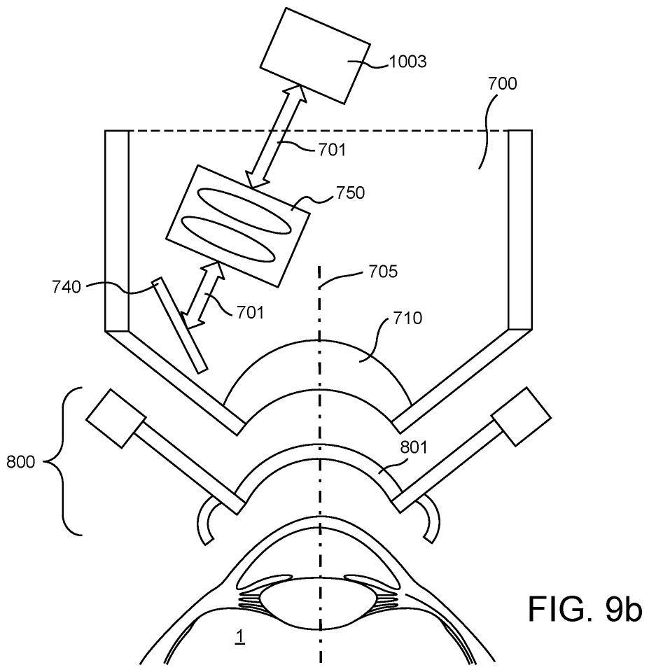

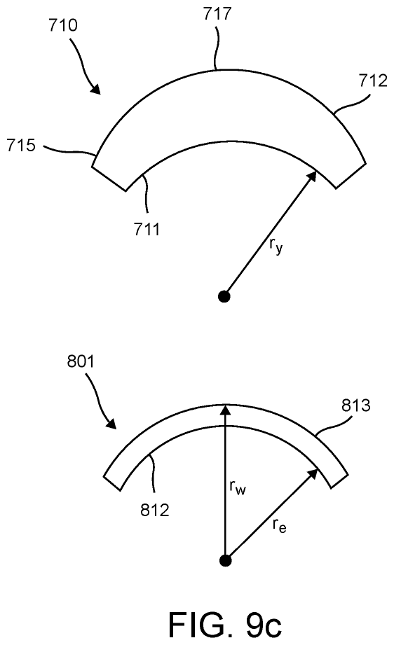

With reference to FIGS. 9a and 9b, in an embodiment of the integrated surgical system 1000, optical components of the focusing objective 700 and patient interface 800 are configured to minimize spatial and chromatic aberrations and spatial and chromatic distortions. FIG. 9a shows a configuration when both the eye 1, the patient interface 800 and the focusing objective 700 all coupled together. FIG. 9b shows a configuration when both the eye 1, the patient interface 800 and the focusing objective 700 all detached from one another.

The patient interface 800 optically and physically couples the eye 1 to the focusing objective 700, which in turn optically couples with other optic components of the integrated surgical system 1000. The patient interface 800 serves multiple functions. It immobilizes the eye relative to components of the integrated surgical system; creates a sterile barrier between the components and the patient; and provides optical access between the eye and the instrument. The patient interface 800 is a sterile, single use disposable device and it is coupled detachably to the eye 1 and to the focusing objective 700 of the integrated surgical system 1000.

The patient interface 800 includes a window 801 having an eye-facing, concave surface 812 and an objective-facing, convex surface 813 opposite the concave surface. The window 801 thus has a meniscus form. With reference to FIG. 9c, the concave surface 812 is characterized by a radius of curvature rₑ, while the convex surface 813 is characterized by a radius of curvature r_{w}. The concave surface 812 is configured to couple to the eye, either through a direct contact or through index matching material, liquid or gel, placed in between the concave surface 812 and the eye 1. The window 801 may be formed of glass and has a refractive index n_{w}. In one embodiment, the window 801 is formed of fused silica and has a refractive index n_{w} of 1.45. Fused silica has the lowest index from common inexpensive glasses. Fluoropolymers such as the Teflon AF are another class of low index materials that have refractive indices lower than fused silica, but their optical quality is inferior to glasses and they are relatively expensive for high volume production. In another embodiment the window 801 is formed of the common glass BK7 and has a refractive index n_{w} of 1.50. A radiation resistant version of this glass, BK7G18 from Schott AG, Mainz, Germany, allows gamma sterilization of the patient interface 800 without the gamma radiation altering the optical properties of the window 801.

Returning to FIGS. 9a and 9b, the window 801 is surrounded by a wall 803 of the patient interface 800 and an immobilization device, such as a suction ring 804. When the suction ring 804 is in contact with the eye 1, an annular cavity 805 is formed between the suction ring and the eye. When vacuum applied to the suction ring 804 and the cavity via a vacuum tube a vacuum pump (not shown in FIGS. 9a and 9b), vacuum forces between the eye and the suction ring attach the eye to the patient interface 800 during surgery. Removing the vacuum releases or detach the eye 1.

The end of the patient interface 800 opposite the eye 1 includes an attachment interface 806 configured to attach to the housing 702 of the focusing objective 700 to thereby affix the position of the eye relative to the other components of the integrated surgical system 1000. The attachment interface 806 can work with mechanical, vacuum, magnetic or other principles and it is also detachable from the integrated surgical system.

The focusing objective 700 includes an aspheric exit lens 710 having an eye-facing, concave surface 711 and a convex surface 712 opposite the concave surface. The exit lens 710 thus has a meniscus form. While the exit lens 710 shown in FIGS. 9a and 9b is an aspheric lens giving more design freedom, in other configurations the exit lens may be a spherical lens. Alternatively, constructing the exit lens 710 as a compound lens, as opposed to a singlet, allows more design freedom to optimize the optics while preserving the main characteristics of the optical system as presented here. With reference to FIG. 9c, the concave surface 711 is characterized by a radius of curvature r_{y}, while the convex surface 712 is characterized by an aspheric shape. The aspheric convex surface 712 in combination with the spherical concave surface 711 result in an exit lens 710 having varying thickness, with the outer perimeter edges 715 of the lens being thinner than the central, apex region 717 of the lens. The concave surface 711 is configured to couple to the convex surface 813 of the window 801. In one embodiment, the exit lens 710 is formed of fused silica and has a refractive index nₓ of 1.45.

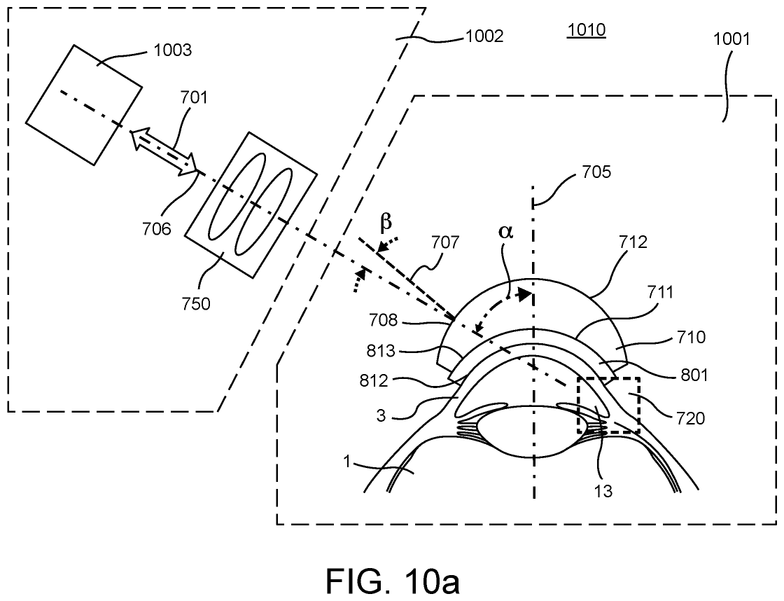

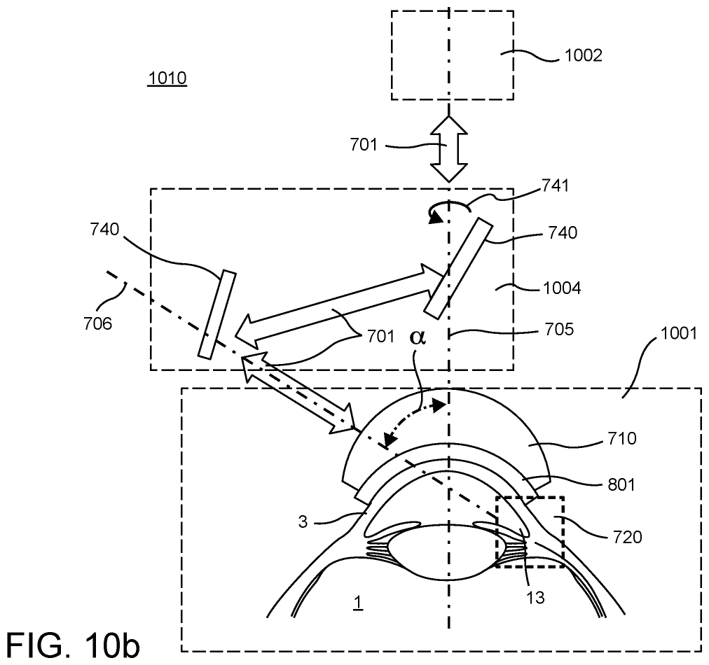

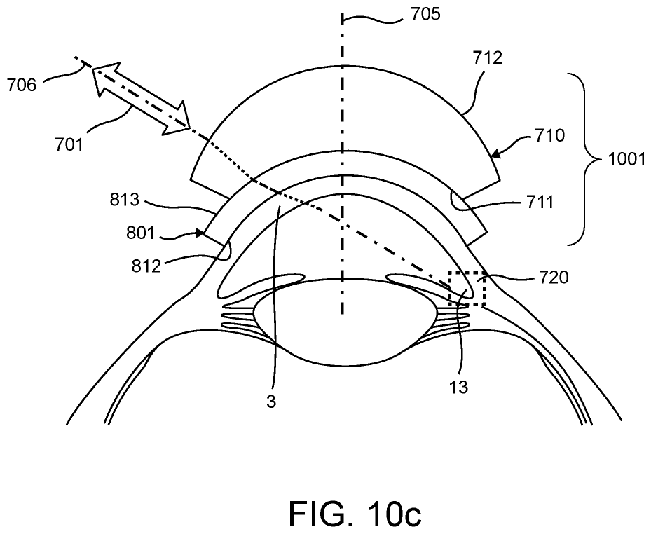

FIGS. 10a and 10b are schematic illustrations of components of the integrated surgical system of FIGS. 7 and 8 functionally arranged to form an optical system 1010 having a first optical subsystem 1001 and a second optical subsystem 1002 that enable access to a surgical volume 720 in the irido-corneal angle. Each of FIGS. 10a and 10b include components of the focusing objective 700 and the patient interface 800 of FIG. 9a. However, for simplicity, the entirety of the focusing objective and the patient interface are not included in FIGS. 10a and 10b. Also, for additional simplicity in FIG. 10a, the planar beam-folding mirror 740 of FIGS. 9a and 9b is not included and the combined laser/OCT/illumination beam 701 shown in FIG. 9a is unfolded or straightened out. It is understood by those skilled in the art that adding or removing planar beam folding mirrors does not alter the principal working of the optical system formed by the first optical subsystem and the second optical subsystem. FIG. 10c is a schematic illustration of a beam passing through the first optical subsystem of FIGS. 10a and 10b.

With reference to FIG. 10a, a first optical subsystem 1001 of the integrated surgical system 1000 includes the exit lens 710 of a focusing objective 700 and the window 801 of a patient interface 800. The exit lens 710 and the window 801 are arranged relative to each other to define a first optical axis 705. The first optical subsystem 1001 is configured to receive a beam, e.g., a combined laser/OCT/illumination beam 701, incident at the convex surface 712 of the exit lens 710 along a second optical axis 706, and to direct the beam toward a surgical volume 720 in the irido-corneal angle 13 of the eye.

During a surgical procedure, the first optical subsystem 1001 may be assembled by interfacing the convex surface 813 of the window 801 with the concave surface 711 of the exit lens 710. To this end, a focusing objective 700 is docked together with a patient interface 800. As a result, the concave surface 711 of the exit lens 710 is coupled to the convex surface 813 of the window 801. The coupling may be by direct contact or through a layer of index matching fluid. For example, when docking the patient interface 800 to focusing objective 700, a drop of index matching fluid can be applied between the contacting surfaces to eliminate any air gap that may be between the two surfaces 711, 813 to thereby help pass the combined laser/OCT/illumination beam 701 through the gap with minimal Fresnel reflection and distortion.

In order to direct the combined laser/OCT/illumination beam 701 toward the surgical volume 720 in the irido-corneal angle 13 of the eye, the first optical subsystem 1001 is designed to account for refraction of the beam 701 as it passes through the exit lens 710, the window 801 and the cornea 3. To this end, and with reference to FIG. 10c, the refractive index nₓ of the exit lens 710 and the refractive index n_{w} of the window 801 are selected in view of the refractive index nₓ of the cornea 3 to cause appropriate beam bending through the first optical subsystem 1001 so that when the combined laser/OCT/illumination beam 701 exits the subsystem and passes through the cornea 3, the beam path is generally aligned to fall within the irido-corneal angle 13.

Continuing with reference to FIG. 10c and beginning with the interface between the window 801 and the cornea 3. Too steep of an angle of incidence at the interface where the combined laser/OCT/illumination beam 701 exits the window 801 and enters the cornea 3, i.e., at the interface between the concave surface 812 of the window and the convex surface of the cornea 3, can create excessive refraction and distortion. To minimize refraction and distortion at this interface, in one embodiment of the first optical subsystem 1001, the refractive index of the window 801 is closely matched to the index of the cornea 3. For example, as describe above with reference to FIGS. 9a and 9b, the window 801 may have a refractive index lower than 1.42 to closely match the cornea 3, which has a refractive index of 1.36.

Excessive refraction and distortion at the interface where the combined laser/OCT/illumination beam 701 exits the window 801 and enters the cornea 3 may be further compensated for by controlling the bending of the beam 701 as it passes through the exit lens 710 and the window 801. To this end, in one embodiment of the first optical subsystem 1001 the index of refraction n_{w} of the window 801 is larger than each of the index of refraction nₓ of the exit lens 710 and the index of refraction n_{c} of the cornea 3. As a result, at the interface where the combined laser/OCT/illumination beam 701 exits the exit lens 710 and enters the window 801, i.e., interface between the concave surface 711 of the exit lens and the convex surface 813 of the window, the beam passes through a refractive index change from high to low that cause the beam to bend in a first direction. Then, at the interface where the combined laser/OCT/illumination beam 701 exits the window 801 and enters the cornea 3, i.e., interface between the concave surface 812 of the exit lens and the convex surface of the cornea, the beam passes through a refractive index change from low to high that cause the beam to bend in a second direction opposite the first direction.

The shape of the window 801 is chosen to be a meniscus lens. As such, the incidence angle of light has similar values on both surfaces 812, 813 of the window 801. The overall effect is that at the convex surface 813 the light bends away from the surface normal and at the concave surface 812 the light bends towards the surface normal. The effect is like when light passes through a plan parallel plate. Refraction on one surface of the plate is compensated by refraction on the other surface a light passing through the plate does not change its direction. Refraction at the entering, convex surface 712 of the exit lens 710 distal to the eye is minimized by setting the curvature of the entering surface such that angle of incidence β of light 701 at the entering surface is close to a surface normal 707 to the entering surface at the intersection point 708.

Here, the exit lens 710, the window 801, and the eye 1 are arranged as an axially symmetric system with a first optical axis 705. In practice, axial symmetry is an approximation because of manufacturing and alignment inaccuracies of the optical components, the natural deviation from symmetry of the eye and the inaccuracy of the alignment of the eye relative to the window 801 and the exit lens 710 in a clinical setting. But, for design and practical purposes the eye 1, the window 801, and the exit lens 710 are considered as an axially symmetric first optical subsystem 1001.

With continued reference to FIG. 10a, a second optical subsystem 1002 is optically coupled to the first optical subsystem 1001 at an angle α relative to the first optical axis 705 of the first optical subsystem 1001. The advantage of this arrangement is that the both optical subsystems 1001, 1002 can be designed at a much lower numerical aperture compared to a system where all optical components are designed on axis with a common optical axis.

The second optical subsystem 1002 includes an objective lens 750 that, as previously described with reference to FIG. 8, generates a conjugate surgical volume 721 of the surgical volume 720 within the eye. The second optical subsystem 1002 includes various other components collectively indicated as an optical subsystem step 1003. Referring to FIG. 8, these components may include a femtosecond laser source 200, an OCT imaging apparatus 300, a visual observation apparatus 400, beam conditioners and scanners 500, and beam combiners 600.

The second optical subsystem 1002 may include mechanical parts (not shown) configured to rotate the entire subsystem around the first optical axis 705 of the first optical subsystem 1001. This allows optical access to the whole 360-degree circumference of the irido-corneal angle 13 of the eye 1.

With reference to FIG. 10b, flexibility in arranging the first and second optical subsystems 1001, 1002, relative to each other may be provided by an optical assembly 1004 interposed between the optical output of the second optical subsystem 1002 and the optical input of the first optical subsystem 1001. In one embodiment, the optical assembly 1004 may include one or more planar beam-folding mirrors 740, prisms (not shown) or optical gratings (not shown) configured to receive the optical output, e.g., combined laser/OCT/illumination beam 701, of the second optical subsystem 1002, change or adjust the direction of the combined laser/OCT/illumination beam, and direct the beam to the optical input of the first optical subsystem 1001 while preserving the angle α between the first optical axis 705 and the second optical axis 706.

In another configuration, the optical assembly 1004 of planar beam-folding mirrors 740 further includes mechanical parts (not shown) configured to rotate the assembly around the first optical axis 705 of the first optical subsystem 1001 while keeping the second optical subsystem 1002 stationary. Accordingly, the second optical axis 706 of the second optical subsystem 1002 can be rotated around the first optical axis 705 of the first optical subsystem 1001. This allows optical access to the whole 360-degree circumference of the irido-corneal angle 13 of the eye 1.

With considerations described above with reference to FIGS. 9a, 9b and 9c, the design of the first optical subsystem 1001 is optimized for angled optical access at an angle α relative to the first optical axis 705 of the first optical subsystem 1001. Optical access at the angle α compensates for optical aberrations of the first optical subsystem 1001. Table 1 shows the result of the optimization at access angle α=72 degrees with Zemax optical design software package. This design is a practical embodiment for image guided femtosecond glaucoma surgery.

TABLE 1

| | Structure | | | Center |
|---|---|---|---|---|
| | and | Refractive | Radius | Thickness |
| Surface | Material | index | [mm] | [mm] |
| concave | Exit lens | 1.45 | −10 | 4.5 |
| surface | 710 of | | | |
| 711, | focusing | | | |
| convex | objective. | | | |
| surface | Fused silica | | | |
| 712 | | | | |
| concave | Window | 1.50 | −10.9 | 1.0 |
| surface | 801 of | | | |
| 812, | patient | | | |
| convex | interface. | | | |
| surface | BK7G18 | | | |
| 813 | | | | |
| 3 | Cornea | 1.36 | −7.83 | 0.54 |
| 8 | Aqueous | 1.32 | −6.53 | 3.5 |
| | humor | | | |
| Target | Ophthalmic | 1.38 | N/A | 0 to 1 |
| | tissue | | | mm |

This design produces diffraction limited focusing of 1030 nm wavelength laser beams and 850 nm wavelength OCT beams with numerical aperture (NA) up to 0.2. In one design, the optical aberrations of the first optical subsystem are compensated to a degree that the Strehl ratio of the first optical subsystem for a beam with numerical aperture larger than 0.15 at the irido-corneal angle is larger than 0.9. In another design, the optical aberrations of the first optical subsystem are partially compensated, the remaining uncompensated aberrations of the first optical system are compensated by the second optical subsystem to a degree that the Strehl ratio of the combined first and second optical subsystem for a beam with numerical aperture larger than 0.15 at the irido-corneal angle is larger than 0.9.

Laser Surgical Patterns and Parameters

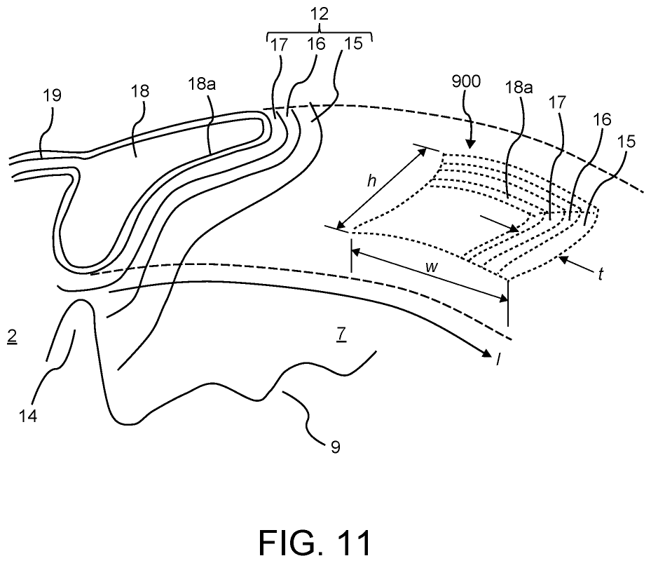

FIG. 11 is a three-dimensional schematic illustration of anatomical structures of the eye relevant to the surgical treatment enabled by the integrated surgical system 1000. To reduce the IOP, laser treatment targets ocular tissues that affect the trabecular outflow pathway 40. These ocular tissues may include the trabecular meshwork 12, the scleral spur 14, the Schlemm's canal 18, and the collector channels 19. The trabecular meshwork 12 has three layers, the uveal meshwork 15, the corneoscleral meshwork 16, and the juxtacanalicular tissue 17. These layers are porous and permeable to aqueous, with the uveal meshwork 15 being the most porous and permeable, followed by the corneoscleral meshwork 16. The least porous and least permeable layer of the trabecular meshwork 12 is the juxtacanalicular tissue 17. The inner wall 18a of the Schlemm's canal 18, which is also porous and permeable to aqueous, has characteristics similar to the juxtacanalicular tissue 17.

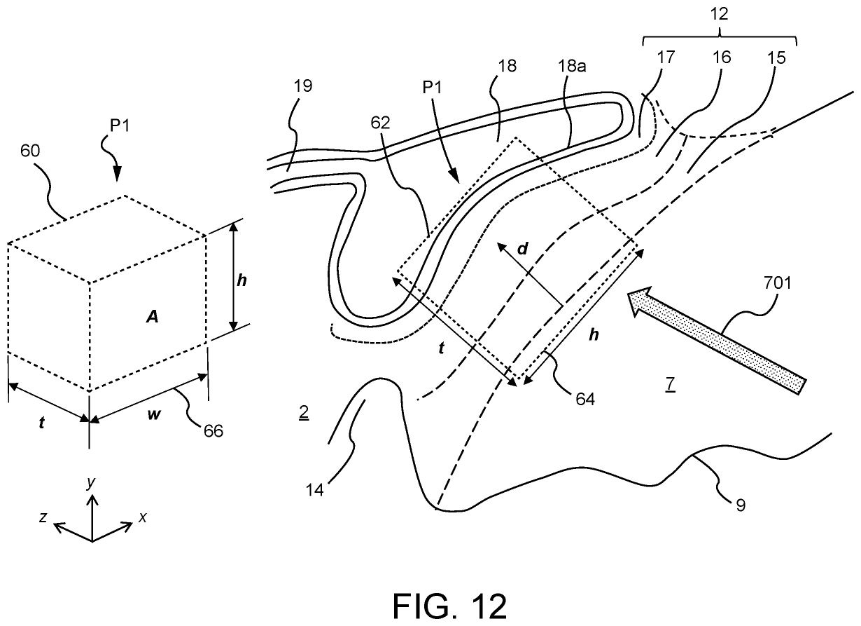

FIG. 12 includes a three-dimensional illustration of a treatment pattern P1 to be applied by the integrated surgical system 1000 to affect the surgical volume 900 of ocular tissue shown in FIG. 11, and a two-dimensional schematic illustration of a treatment pattern P1 overlaying the anatomical structures to be treated. The OCT imaging apparatus 300 of the integrated surgical system 1000 may present a visual image of the anatomical structures similar to the two-dimensional schematic illustration in FIG. 12.

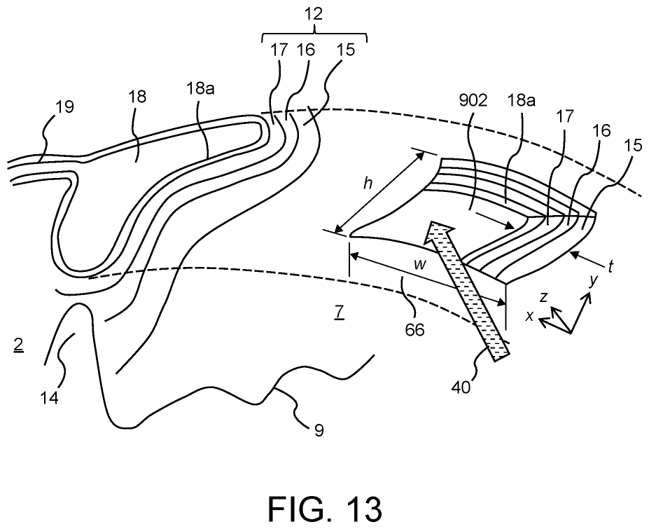

FIG. 13 is a three-dimensional schematic illustration of the anatomical structures of the eye including an opening 902 through the trabecular meshwork 12 and the inner wall 18a of the Schlemm's canal 18 that results from the application of the laser treatment pattern P1 of FIG. 12. The opening 902 resembles the surgical volume 900 and provides a trabecular outflow pathway 40 that reduces the flow resistance in the ocular tissue to increase aqueous flow from the anterior chamber 7 into the Schlemm's canal 18 and thereby reduce the IOP of the eye. The opening 902 may be a continuous, single lumen defining a fluid pathway, or may be defined by an arrangement of adjacent pores forming a sponge like structure defining a fluid pathway or a combination thereof.

The treatment pattern P1 defines a laser scanning procedure whereby a laser is focused at different depth locations in ocular tissue and then scanned in multiple directions to affect a three-dimensional volume of tissue comprising multiple sheets or layers of affected tissue. A treatment pattern is considered to define a collection of a laser-tissue interaction volumes, referred to herein as cells. The size of a cell is determined by the extent of the influence of the laser-tissue interaction. When the laser cells are spaced close along a line, the laser creates a narrow, microscopic channel. A wider channel can be created by closely spacing a multitude of laser cells within the cross section of the channel. The arrangement of the cells may resemble the arrangement of atoms in a crystal structure.

A treatment pattern P1 may be in the form of a cubic structure that encompasses individual cells arranged in regularly spaced rows, columns and sheets or layers. The treatment pattern P1 may be characterized by x, y, z dimensions, with x, y, z coordinates of the cells being calculated sequentially from neighbor to neighbor in the order of a column location (x coordinate), a row location (y coordinate), and a layer location (z coordinate). A treatment pattern P1 as such, defines a three-dimensional model of ocular tissue to be modified by a laser or a three-dimensional model of ocular fluid to be affected by a laser.

A treatment pattern P1 is typically defined by a set of surgical parameters. The surgical parameters may include one or more of a treatment area A that represents a surface area or layer of ocular tissue through which the laser will travel. The treatment area A is determined by the treatment height, h, and the width or lateral extent 66 of the treatment, w. The lateral extent 66 may be defined in terms of a measure around the circumferential angle. For example, the lateral extent 66 w may be defined in terms of an angle, e.g., 90 degrees, around the circumferential angle. A treatment thickness t that represents the level to which the laser will cut into the ocular tissue from the distal extent 62 or border of the treatment volume at or near the inner wall 18a of the Schlemm's canal 18 to the proximal extent 64 or border at or near the surface of the trabecular meshwork 12. Thus, a laser applied in accordance with a treatment pattern may affect or produce a surgical volume that resembles the three-dimensional model of the treatment pattern, or may affect fluid located in an interior of an eye structure resembled by the three-dimensional model.

Additional surgical parameters define the placement of the surgical volume or affected volume within the eye. For example, with reference to FIGS. 11 and 12, placement parameters may include one or more of a location 1 that represents where the treatment is to occur relative to the circumferential angle of the eye, and a treatment depth d that represents a position of the three-dimensional model of ocular tissue or ocular fluid within the eye relative to a reference eye structure. In the following, the treatment depth d is shown and described relative to the region where the anterior chamber 7 meets the trabecular meshwork 12. Together, the treatment pattern and the placement parameters define a treatment plan.

As previously mentioned, the laser treatment described herein involves photodisruption by a femtosecond laser. A femtosecond laser provides highly localized, non-thermal photo-disruptive laser-tissue interaction with minimal collateral damage to surrounding ocular tissue. Photo-disruptive interaction of the laser is utilized in optically transparent tissue. The principal mechanism of laser energy deposition into the ocular tissue is not by absorption but by a highly nonlinear multiphoton process. This process is effective only at the focus of the pulsed laser where the peak intensity is high. Regions where the beam is traversed but not at the focus are not affected by the laser. Therefore, the interaction region with the ocular tissue is highly localized both transversally and axially along the laser beam.

During a laser scanning procedure, a laser focus is moved to different depths d in ocular tissue and then scanned in two lateral dimensions or directions as defined by a treatment pattern P1 to affect a three-dimensional volume 900 of ocular tissue comprising multiple sheets or layers of affected tissue. The two lateral dimensions are generally orthogonal to the axis of movement of the laser focus. With reference to FIG. 13, the movement of a laser focus during laser scanning is described herein with reference to x, y, and z directions or axes. Movement of the laser focus to different depths d through the thickness t of treatment pattern P1 or the volume 900 of tissue corresponds to movement of the focus along the z axis. The focal point of the laser in the z direction may be referred to as a depth d within the treatment pattern P1 or the volume 900 of tissue.

Movement of the laser focus in two dimensions or directions orthogonal to the z axis corresponds to movement of the laser focus along the width w of the treatment pattern P1 or the volume 900 of tissue in the x direction, and movement of the laser focus along the height h of the treatment pattern P1 or the volume 900 of tissue in the y direction. The two direction or dimension scanning of the laser focus may be in the form of a raster scan and defines a layer of laser scanning, which in turn produces a layer of laser-affected tissue.

During laser scanning, pulse shots of a laser are delivered to tissue within the volume of ocular tissue corresponding to the treatment pattern P1. Because the laser interaction volume is small, on the order of a few micrometers (μm), the interaction of ocular tissue with each laser shot of a repetitive laser breaks down ocular tissue locally at the focus of the laser. Pulse duration of the laser for photo-disruptive interaction in ocular tissue can range from several femtoseconds to several nanoseconds and pulse energies from several nanojoules to tens of microjoules. The laser pulses at the focus, through multiphoton processes, breaks down chemical bonds in the molecules, locally photo-dissociate tissue material and create gas bubbles in wet tissue. The breakdown of tissue material and mechanical stress from bubble formation fragments the tissue and create clean continuous cuts when the laser pulses are laid down in proximity to one another along geometrical lines and surfaces.

Laser Surgical Treatment with Second Harmonic Light Detection

As noted above, a femtosecond laser provides highly localized laser-tissue interaction that create a cutting effect in tissue at the focus of the femtosecond laser beam. During a laser treatment procedure, surgical femtosecond lasers are tightly focused to a spot at a predetermined location distal to the focusing optics or objective of the surgical system. Having created such a focus and prior to beginning a laser treatment, it is desirable to monitor the location of the focus relative to a structure of ocular tissue, to accurately locate the focus with respect to the target structure, and to avoid locating the focus at tissue that should not be treated.

The second harmonic light detection apparatus 450 described above may be used to detect or locate the distal extent 62 of a target volume of ocular tissue, or the proximal extent 64 of the target volume, or an interior of a target structure. To this end, the second harmonic light detection apparatus 450 is configured to detect a second harmonic light that results from an encounter between the focus of the femtosecond laser beam 701 and tissue. When the focus of the laser 701 is not encountering tissue, a spot of second harmonic light is not present or if present, has an intensity level or measurement in a lower range. When the focus of the femtosecond laser beam 701 is encountering tissue, a spot of second harmonic light having an intensity level or measurement in an upper range is present. Based on this, a distal extent 62 such as shown in FIG. 12 may be detected by first advancing the focus of the femtosecond laser beam 701 through the trabecular meshwork 12 and the inner wall 18a of the Schlemm's canal 18, at which times the focus will encounter tissue and a spot of second harmonic light will be present, and into the Schlemm's canal, where the focus will no longer encounter tissue and a spot of second harmonic light will not be present, and then retracting the focus of the laser 701 back toward the inner wall of the Schlemm's canal and detecting that the focus is at the inner wall when a spot of second harmonic light is present.

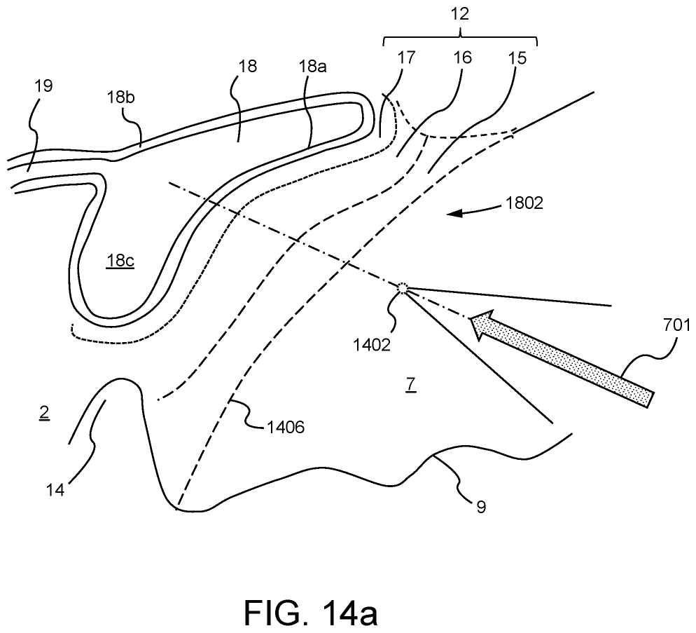

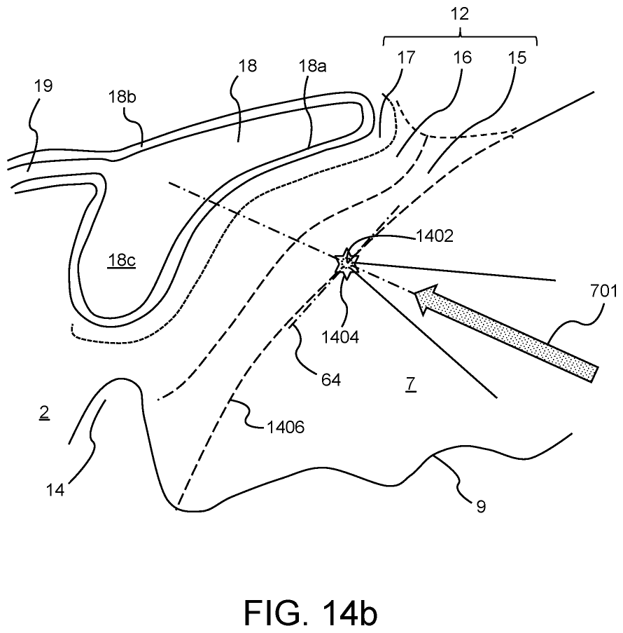

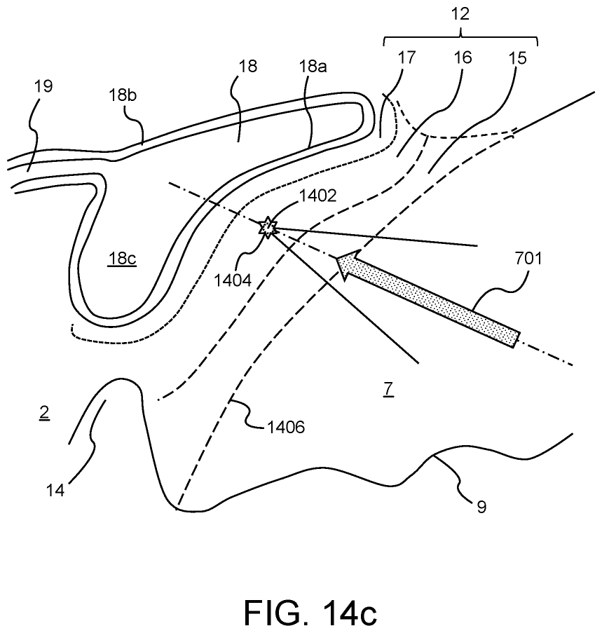

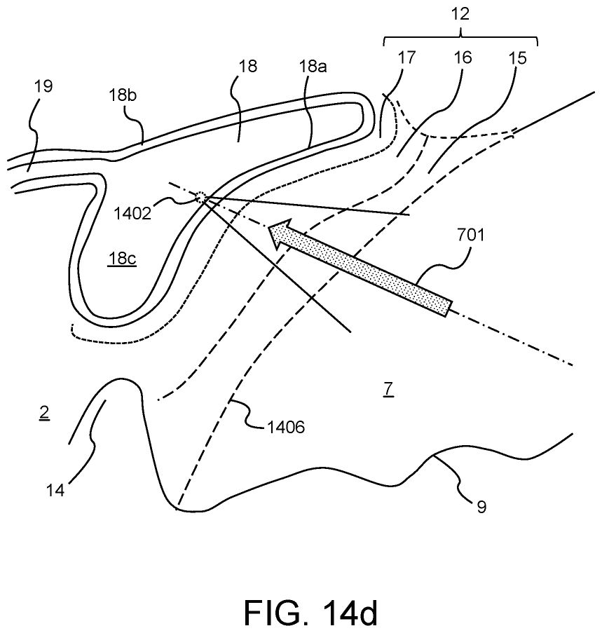

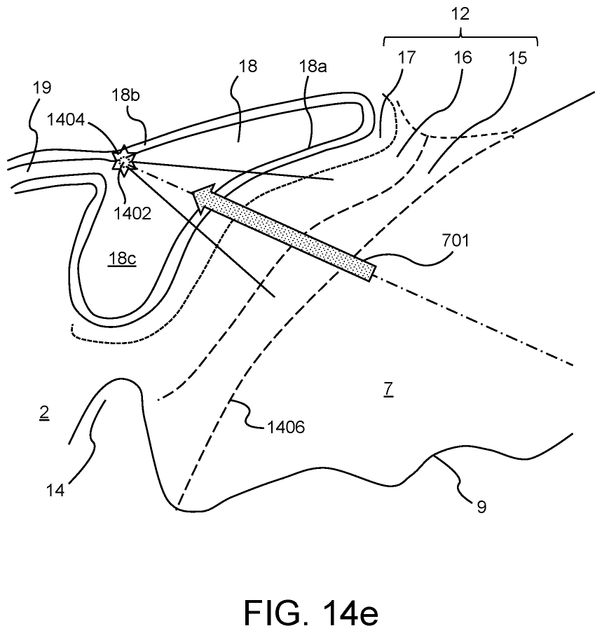

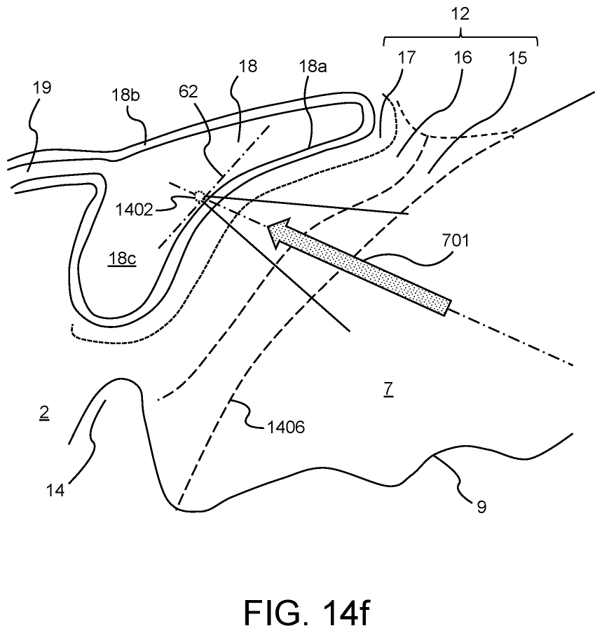

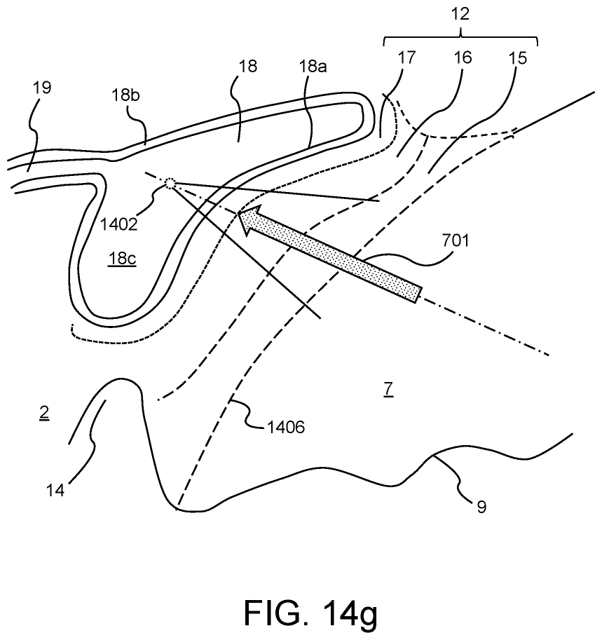

With reference to FIGS. 14a-14g, in accordance with embodiments disclosed herein a target surface corresponding to a distal extent 62 of a target volume of ocular tissue in an irido-corneal angle of an eye is located by a femtosecond laser. To this end, the focus 1402 of the femtosecond laser beam 701 is advanced, in the direction of propagation, through ocular tissue to a location in the irido-corneal angle. The location is at or near the target surface 62. During advancement, changes in an intensity of a spot 1404 of second harmonic light generated by an encounter between the focus 1402 of the femtosecond laser beam 701 and tissue are monitored to determine when the focus is at the target surface 62. During advancement of the focus 1402, the femtosecond laser it set to a power level insufficient to photodisrupt tissue.

With reference to FIGS. 7, 8 and 14a, a patient's eye 1 is coupled through a patient interface 800 to a focusing objective 700 of an integrated surgical system 1000. A second harmonic light detection apparatus 450 detects a second harmonic light beam 451, if any, present in the irido-corneal angle of the eye 1, and depending on its configuration, may also provide an image of the tissue structures of the irido-corneal angle. In one embodiment, the second harmonic light detection apparatus 450 may be a photodetector that detects a spot of second harmonic light and outputs a signal indicating a presence of such spot when a level or measure of SHG intensity is in a range indicative of a presence of second harmonic light. For example, with reference to FIG. 17, a spot of second harmonic light may be detected, e.g., determined to be present, when the SHG intensity is in a high-level range 1708, 1716. Conversely, a spot of second harmonic light may be undetected, e.g., determined to be absent, when the SHG intensity is outside of an high-level range 1708, 1716. Alternatively, the second harmonic light detection apparatus 450, e.g., the photodetector, may output for display on the control system 100 a SHG intensity profile, such as shown in FIG. 17, for observation and interpretation by a surgeon. The SHG intensity profile may be output in real time to guide a surgeon during laser surgery.

In another embodiment, the second harmonic light detection apparatus 450 may be a video camera that captures an image of the irido-corneal angle of the eye 1 and either displays the image for viewing by a surgeon or processes the image to determine the presence or absence of a spot of second harmonic light. Similar to the photodetector embodiment, the presence or absence of a spot of second harmonic light may be determine based on a level or measure of SHG intensity. In this case, however, the level or measure of SHG intensity is based on image processing of the video images. For example, the control system 100 may be configured to analyze the video image to detect a spot of second harmonic light and to determine a level or measure of intensity of the spot based on the brightness of the spot.

Referring to FIG. 14a, the focus 1402 of the femtosecond laser beam 701 is initially in the anterior chamber 7 some distance from an initial surface 1406 of ocular tissue. At this stage, the symmetry of the material, e.g., the void in the anterior chamber, in the region of the focus 1402 of the femtosecond laser beam 701 is isotropic and no spot of second harmonic light is present.

With reference to FIG. 14b, the focus 1402 of the femtosecond laser beam 701 is moved inside the eye 1 in the direction of propagation of the femtosecond laser beam towards the trabecular meshwork 12. The focus 1402 may be moved by the control system 100 under operation of a surgeon. When the focus 1402 encounters the initial surface 1406 of ocular tissue, in this case, a surface of the uveal meshwork 15, the symmetry of the light of the laser is broken and second harmonic generation is created at the interface between the focus and the initial surface. A spot 1404 of second harmonic light results from the surface second harmonic generation and is observable through the video camera of the second harmonic light detection apparatus 450. The generation of this light marks the initial surface of the trabecular meshwork 12. This location may define the location of the proximal extent 64 of the volume of ocular tissue to be treated.

The color of the spot 1404 of second harmonic light is a function of the wavelength of the femtosecond laser. In one configuration, the wavelength of the femtosecond laser is 1030 nm (infrared) and the wavelength of the second harmonic light is 515 nm (green). Accordingly, the spot 1404 of second harmonic light appears as a green spot.

With reference to FIG. 14c, the focus 1402 of the femtosecond laser beam 701 is moved in the direction of propagation of the femtosecond laser beam further into the trabecular meshwork 12. The focus 1402 may be moved by the control system 100 under operation of a surgeon. During this movement of the focus 1402, the intensity of the spot 1404 of second harmonic light may vary as the focus 1402 encounters the tissue fibers that comprise the layers of the trabecular meshwork 12, i.e., the uveal meshwork 15, the corneoscleral meshwork 16, and the juxtacanalicular tissue 17.

With reference to FIG. 14d, the focus 1402 of the femtosecond laser beam 701 is again moved in the direction of propagation of the femtosecond laser beam through the trabecular meshwork 12 and the inner wall 18a of the Schlemm's canal. During this movement of the focus 1402, the intensity of the spot 1404 of second harmonic light may diminish as it passes through the trabecular meshwork 12 and the inner wall 18a of the Schlemm's canal, until it disappears. The depth d at which the spot 1404 of second harmonic light disappears marks the transition from the juxtacanalicular tissue 17 of the trabecular meshwork 12 to the interior 18c of the Schlemm's canal 18. This depth d may be measured relative to the initial surface 1406 of ocular tissue.

With reference to FIG. 14e, the focus 1402 of the femtosecond laser beam 701 is again moved in the direction of propagation of the femtosecond laser beam through the interior of the Schlemm's canal 18 toward the outer wall 18b of the Schlemm's canal. During this movement of the focus 1402, the spot 1404 of second harmonic light eventually reappears when the focus 1402 encounters the tissue fibers that comprise the outer wall 18b of the Schlemm's canal. The depth d at which the spot 1404 of second harmonic light reappears marks the outer wall 18b of the Schlemm's canal 18.

Using the respective depths of the inner wall 18a and the outer wall 18b of the Schlemm's canal 18, the integrated surgical system 1000 may calculate the width of the Schlemm's canal 18 along the propagation path of the femtosecond laser beam 701. The width of the Schlemm's canal 18 informs the system or surgeon using the system of an appropriate location, e.g., the inner wall 18a of the Schlemm's canal, of the focus 1402 for initiation of laser treatment, and the relative risk of photodisrupting the outer wall 18b of the Schlemm's canal during such treatment. For example, if the inner wall 18a and the outer wall 18b of the Schlemm's canal 18 are too close, e.g., a width of 20 to 50 microns, then the laser energy delivered to the inner wall 18a during treatment may also photodisrupt the outer wall 18b, which is to be avoided. In such cases, the integrated surgical system 1000 may notify the surgeon of the possibility of photodisrupting the outer wall 18b or may, as a safety feature, automatically prevent the initiation of photodisruption.

With reference to FIG. 14f, the focus 1402 of the femtosecond laser beam 701 is moved in a direction opposite the direction of propagation of the femtosecond laser beam, away from the outer wall 18b of the Schlemm's canal and through the interior 18c of the Schlemm's canal 18 toward the inner wall 18a of the Schlemm's canal. To this end, the focus 1402 may be moved back to the previously determined depth d that marks the transition from the juxtacanalicular tissue 17 of the trabecular meshwork 12 to the interior of the Schlemm's canal 18. This location at depth d may define the location of the distal extent 62 of the volume of ocular tissue to be treated.

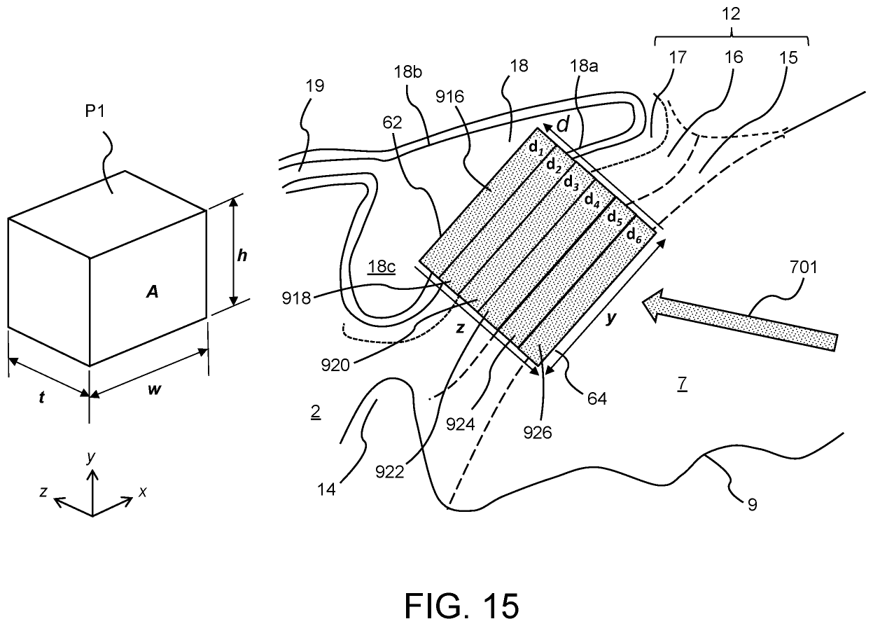

With reference to FIG. 15, having determined the location of the distal extent 62 of the volume of ocular tissue to be treated, the power of the femtosecond laser may be increased to a level sufficient to photodisrupt ocular tissue and the volume of ocular tissue between the distal extent 62 and the proximal extent 64 may be treated. To this end, during a laser scanning procedure in accordance with treatment pattern P1, the focus of the femtosecond laser beam 701 is initially located at the depth d₁. This depth d₁ places the laser focus in an initial layer 916 of tissue near the inner wall 18a of the Schlemm's canal 18, but a safe distance from the outer wall 18b. Once the laser focus is positioned at the initial depth d₁, the focus may be raster scanned, with optical energy being delivered at periodic instances in time during the scan. These instances in time where optical energy is delivered to tissue result in the photodisruption of the initial layer 916 of tissue and define an initial treatment plane at the initial layer of tissue.

With continued reference to FIG. 15, the focus of the femtosecond laser beam 701 is then moved in the z direction toward the anterior chamber 7 to a subsequent depth d₂. The subsequent depth d₂ places the laser focus at a subsequent layer 918 of tissue. Once the laser focus is positioned at the subsequent depth d₂, the focus is raster scanned, with optical energy being delivered at instances in time during the scan. These instances of optical energy delivery result in the photodisruption of the subsequent layer 918 of tissue and define a subsequent treatment plane at the subsequent layer of tissue. The foregoing movement of the focus of the femtosecond laser beam 701 and laser scanning and optical energy delivery is repeated at depths d₃, d₄, d₅, and d₆ resulting in photodisruption of the subsequent layers 918, 920, 922, 924, 926 of tissue.

With reference to FIG. 13, photodisruption of the multiple layers forms an opening 902 between the anterior chamber 7 and the Schlemm's canal 18, thus completing the laser treatment procedure. The opening 902 resulting from laser application of the treatment pattern P1 resembles the surgical volume 900 and is characterized by an area A and thickness t similar to those of the surgical volume and the treatment pattern. The thickness t of the resulting opening 902 extends from the anterior chamber 7 and through the inner wall 18a of the Schlemm's canal 18, while the area A defines the cross-section size of the opening 902.

In other laser treatments, instead of locating the focus 1402 of the femtosecond laser beam 701 in ocular tissue, it may be desirable to locate the focus within a structure. For example, as disclosed in U.S. patent application Ser. No. 16/125,588, entitled Non-Invasive and Minimally Invasive Laser Surgery for the Reduction Of Intraocular Pressure in the Eye, the disclosure of which is hereby incorporated by reference, pneumatic expansion may be utilized to open collapsed regions of the Schlemm's canal 18 and collector channels 19 and in general increase outflow for an IOP reducing effect. In such laser treatment, the fluid inside the Schlemm's canal 18 or the collector channels 19 is photodisrupted.

Accordingly, with reference to FIG. 14g the focus 1402 of the femtosecond laser beam 701 may be moved in a direction opposite the direction of propagation of the femtosecond laser beam, away from the outer wall 18b of the Schlemm's canal to a location in the interior 18c of the Schlemm's canal 18. To this end, the focus 1402 may be moved back toward the inner wall 18a of the Schlemm's canal a distance based on the previously determined depth d, e.g., one half the depth, obtained as described above with reference to FIG. 14d, or the focus may be moved based on the width of the Schlemm's canal 18, e.g., one half the width, obtained as described above with reference to FIG. 14e.

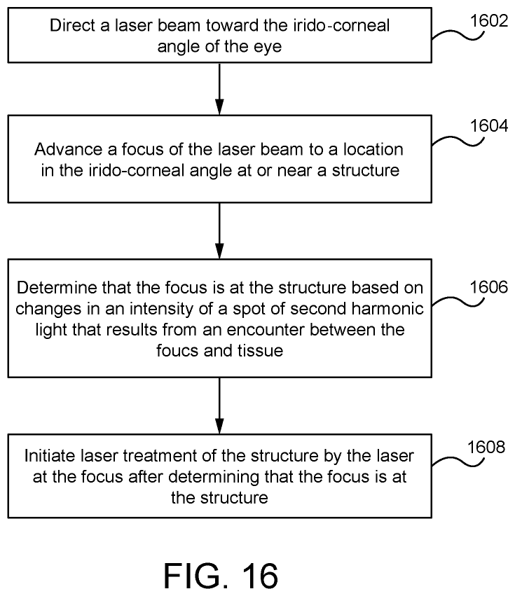

FIG. 16 is a flowchart of a method of locating a target structure in an irido-corneal angle of an eye based on second harmonic light generation that results from an encounter between a focus of a laser and tissue. The target structure may be a surface of ocular tissue, such as the inner wall 18a of the Schlemm's canal 18, the outer wall 18b of Schlemm's canal, or a proximal surface of the trabecular meshwork 12. The target structure may also be an interior of a structure, such as the interior 18c of the Schlemm's canal 18. The laser is configured to produce optical pulses of sufficiently short duration, e.g., 1 nanosecond or less. These short duration pulses enable the generation of second harmonic light. They also enable the photodisruption of ocular tissue, provided the power level of the pulses is at a sufficient level. In the description to follow, the laser is a femtosecond laser. Other lasers, however, may be used; including for example, a Q-switched Nd:YAG laser.

The method, which may be performed by the integrated surgical system 1000 of FIGS. 7-10b, begins at a point in a surgical procedure where access to the irido-corneal angle has been obtained. Systems and methods for accessing the irido-corneal angle are described in U.S. patent application Ser. No. 16/036,883, entitled Integrated Surgical System and Method for Treatment in the Irido-Corneal Angle of the Eye, the disclosure of which is hereby incorporated by reference.

At block 1602, the integrated surgical system 1000 directs a femtosecond laser beam 701 toward the irido-corneal angle of the eye 1. The femtosecond laser beam 701 is directed in a direction of propagation of the laser. The femtosecond laser is maintained at a power level insufficient to photodisrupt tissue while it is being directed toward the irido-corneal angle, and is typically maintained at this power level until the time laser treatment of tissue is initiated.

At block 1604, the integrated surgical system 1000 advances a focus 1402 of the femtosecond laser beam 701 to a location in the irido-corneal angle. The direction of advancement of the focus 1402 is generally in the direction of propagation of the femtosecond laser beam 701, but may alternatively be in a direction opposite the direction of propagation of the laser beam. For example, assuming the location of the focus 1402 is in the anterior chamber 7 at the start of the method, the focus would be advanced in the direction of propagation of the femtosecond laser beam 701 However, if the focus 1402 happens to be in the interior 18c of the Schlemm's canal 18 at the start of the method, the focus would be advanced in the direction opposite the direction of propagation of the laser beam. The location to which the focus 1402 is advanced is at or near the target structure.

At block 1606, the integrated surgical system 1000 determines that the focus 1402 is at the target structure based on changes in an intensity of a spot 1404 of second harmonic light that results from an encounter between the focus and tissue.

The integrated surgical system 1000 may determine that the focus 1402 is as the target surface by monitoring changes in the intensity of second harmonic light. Such monitoring may include detecting a plurality of intensity states of a spot 1404 of second harmonic light as the focus 1402 of the femtosecond laser beam is advanced and detecting a particular sequence or pattern of the plurality of detected intensity states that maps to the target structure. A detected intensity state of a spot 1404 of second harmonic light may correspond to an appearance or presence of a spot of second harmonic light or a disappearance or absence of a spot of second harmonic light.

To this end, in one embodiment, the second harmonic light detection apparatus 450 included in the integrated surgical system 1000 may be a photodetector that detects a spot of second harmonic light and outputs a signal indicating a presence of such spot when a level or measure of SHG intensity is in a range indicative of a presence of second harmonic light, or an absence of such spot otherwise, as previously described with reference to FIG. 17. A processor of the control system 100 may receive the signal from the photodetector, track the spot present/absent pattern represented by the signal, and compare the pattern to a look up table that maps different patterns to different target structures. Alternatively, the photodetector may output for display on the control system 100 a SHG intensity profile, such as shown in FIG. 17, for observation and interpretation by a surgeon.

In another embodiment, the second harmonic light detection apparatus 450 included in the integrated surgical system 1000 may be a video camera that captures an image of the irido-corneal angle of the eye 1 and displays the image for viewing by a surgeon and/or transmits the image to the control system 100 for processing to determine the presence or absence of a spot of second harmonic light. Similar to the photodetector embodiment, the presence or absence of a spot of second harmonic light may be determined based on a level or measure of SHG intensity. In this case, however, a processor of the control system 100 is configured to execute image processing algorithms to recognize the presence of a spot 1404 of second harmonic light in an image. Further, the processor is configured to determine the brightness or intensity of the spot 1404 in the image and to correlate the determined brightness to a level or measure of SHG intensity. Using this information, the processor of the control system 100 tracks the present/absent pattern of the spot represented by the image, and compares the pattern to a look up table that maps different patterns to different target structures.

In one example and with reference to FIGS. 14a and 14b, the target structure may be a proximal surface 1406 of a trabecular meshwork 12. The integrated surgical system 1000 determines that the focus 1402 is at the proximal surface 1406 of a trabecular meshwork 12 by detecting a pattern of intensity states that includes: an absence of a spot 1404 of second harmonic light (FIG. 14a), followed by a presence of a spot 1404 of second harmonic light (FIG. 14b). As previously described with reference to FIG. 17, absence of a spot of second harmonic light may correspond to either a complete and total lack of generation of a second harmonic light or a generation of a second harmonic light having a level or measure of intensity outside of a range indicative of a presence of spot of second harmonic light. Conversely, a presence of a spot of second harmonic light may correspond to a generation of a second harmonic light having a level or measure of intensity within a range indicative of a presence of spot of second harmonic light. A present second harmonic light may have different levels of intensity depending on the tissue it encounters.

In another example and with reference to FIGS. 14a, 14b, and 14d, the target structure may be the inner wall 18a of the Schlemm's canal 18. In one embodiment, the integrated surgical system 1000 determines that the focus 1402 is at the inner wall 18a of the Schlemm's canal 18 by detecting a pattern of intensity states that includes: a first absence of a spot 1404 of second harmonic light (FIG. 14a), followed by a presence of a spot 1404 of second harmonic light (FIG. 14b), followed by a second absence of a spot 1404 of second harmonic light (FIG. 14d). In another embodiment and with reference to FIGS. 14a, 14b, 14d, 14e, and 14f, the integrated surgical system 1000 determines that the focus 1402 is at the inner wall 18a of the Schlemm's canal 18 by detecting a pattern of intensity states that includes: a first absence of a spot 1404 of second harmonic light (FIG. 14a), followed by a first presence of a spot 1404 of second harmonic light (FIG. 14b), followed by a second absence of a spot 1404 of second harmonic light (FIG. 14d), followed by a second presence of a spot 1404 of second harmonic light (FIG. 14e), followed by a third absence of a spot 1404 of second harmonic light (FIG. 14f). In this embodiment, the second presence of a spot 1404 of second harmonic light (FIG. 14e) represents a focus 1402 location at an outer wall 18b of the Schlemm's canal 18 and the third absence of a spot 1404 of second harmonic (FIG. 140 represents a focus 1402 location at the inner wall 18a of the Schlemm's canal 18 that results from retracting the focus 1402 of femtosecond laser beam 701 a predetermined distance in the direction opposite the direction of propagation of the laser. The predetermined distance may be based on or correspond to the depth d obtained as described above with reference to FIG. 14d or it may be based on or correspond to the width of the Schlemm's canal 18 obtained as described above with reference to FIG. 14e.

In another example and with reference to FIGS. 14a, 14b, 14c, 14d, 14g, the target structure may be the interior 18c of the Schlemm's canal 18. The integrated surgical system 1000 determines that the focus 1402 is at the interior 18c of the Schlemm's canal 18 by detecting a pattern of intensity states that includes: a first absence of a spot 1404 of second harmonic light (FIG. 14a), followed by a first presence of a spot 1404 of second harmonic light (FIG. 14b), followed by a second absence of a spot 1404 of second harmonic light (FIG. 14d), followed by a second presence of a spot 1404 of second harmonic light (FIG. 14e), followed by a third absence of a spot 1404 of second harmonic light (FIG. 14g). In this embodiment, the second presence of a spot 1404 of second harmonic light (FIG. 14e) represents a focus 1402 location at an outer wall 18b of the Schlemm's canal 18 and the third absence of a spot 1404 of second harmonic (FIG. 14g) represents a focus 1402 location at the interior 18c of the Schlemm's canal 18 that results from retracting the focus 1402 of femtosecond laser beam 701 a predetermined distance in the direction opposite the direction of propagation of the laser. The predetermined distance may be based on the depth d, e.g., one half the depth, obtained as described above with reference to FIG. 14d or it may be based on the width of the Schlemm's canal 18, e.g., one half the width, obtained as described above with reference to FIG. 14e.

Returning to FIG. 16, at block 1608, after determining that the focus is at the target structure, the integrated surgical system 1000 initiates laser treatment of the target structure by the femtosecond laser at the focus 1402. To this end, the power level of the femtosecond laser is increased to a power level sufficient to photodisrupt tissue and the focus is raster scanned in two dimensions to photodisrupt a sheet or layer of tissue.

With reference to FIGS. 7-10b, a surgical system 1000 for implementing the method of FIG. 16 includes a focusing objective 700 configured to be coupled to an eye 1. The focusing objective 700 is aligned to receive a femtosecond laser beam 701 and to direct the femtosecond laser beam toward the irido-corneal angle of the eye. The surgical system 1000 also includes a laser subsystem 1006 that includes a femtosecond laser source 200, a beam conditioner and scanner 500, and beam combiners 600. The laser subsystem 1006 is configured to output the femtosecond laser beam 701 to the focusing objective 700. The surgical system 1000 further includes a second harmonic light detection apparatus 450 that is configured to capture an image of a spot 1404 of second harmonic light generated by an encounter between a focus 1402 of the femtosecond laser beam 701 and tissue.

The surgical system 1000 further includes a control system 100 that is coupled to the laser subsystem 1006 and the second harmonic light detection apparatus 450. The control system 100 is configured to advance the focus 1402 of the femtosecond laser beam 701 to a location in the irido-corneal angle that is at or near the target structure. The control system 100 is also configured to determine that the focus 1402 is at or near the target structure based on changes in an intensity of the spot 1404 of second harmonic light. To this end, the control system 100 is configured to detect a plurality of intensity states of the spot 1404 of second harmonic light as the focus 1402 of the femtosecond laser beam 701 is advanced. Each of the plurality of intensity states corresponds to one of a presence of the spot 1404 of second harmonic light, an absence of the spot 1404 of second harmonic light, or a change in intensity level of the spot 1404 of second harmonic light. The plurality of intensity states define a pattern that maps to the target structure. Thus, upon detection of the pattern, the control system 100 may conclude that the focus 1402 is at or near the target structure, and laser treatment is initiated.

The various aspects of this disclosure are provided to enable one of ordinary skill in the art to practice the present invention. Various modifications to exemplary embodiments presented throughout this disclosure will be readily apparent to those skilled in the art. Thus, the claims are not intended to be limited to the various aspects of this disclosure but are to be accorded the full scope consistent with the language of the claims. All structural and functional equivalents to the various components of the exemplary embodiments described throughout this disclosure that are known or later come to be known to those of ordinary skill in the art are expressly incorporated herein by reference and are intended to be encompassed by the claims. Moreover, nothing disclosed herein is intended to be dedicated to the public regardless of whether such disclosure is explicitly recited in the claims. No claim element is to be construed under the provisions of 35 U.S.C. § 112, sixth paragraph, unless the element is expressly recited using the phrase “means for” or, in the case of a method claim, the element is recited using the phrase “step for.”

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A method of locating a structure in an irido-corneal angle of an eye, the method comprising:
directing a laser beam toward the irido-corneal angle of the eye;
advancing a focus of the laser beam to a location in the irido-corneal angle at or near the structure; and
determining that the focus is at or near the structure based on changes in an intensity of a spot of second harmonic light generated by an encounter between the focus and tissue.

2. The method of claim 1, wherein the determining comprises:
detecting a plurality of intensity states of the spot of second harmonic light as the focus of the laser beam is advanced, wherein each of the plurality of intensity states corresponds to one of a presence of the spot of second harmonic light or an absence of the spot of second harmonic light; and
detecting a pattern of the plurality of intensity states that maps to the structure.

3. The method of claim 2, wherein the structure comprises a proximal surface of a trabecular meshwork and the detected pattern corresponds to an absence of the spot of second harmonic light followed by a presence of the spot of second harmonic light.

4. The method of claim 2, wherein the structure comprises an inner wall of a Schlemm's canal and the detected pattern corresponds to an absence of the spot of second harmonic light followed by a presence of the spot of second harmonic light followed by an absence of the spot of second harmonic light.

5. The method of claim 2, wherein the structure comprises an outer wall of a Schlemm's canal and the detected pattern corresponds to an absence of the spot of second harmonic light followed by a presence of the spot of second harmonic light followed by an absence of the spot of second harmonic light followed by a presence of the spot of second harmonic light.

6. The method of claim 2, wherein the structure comprises an interior of a Schlemm's canal and the detected pattern corresponds to an absence of the spot of second harmonic light followed by a presence of the spot of second harmonic light followed by an absence of the spot of second harmonic light.

7. The method of claim 1, wherein the laser beam is characterized by pulses having a duration of 1 nanosecond or less.

8. The method of claim 7, wherein the laser beam corresponds to one of a femtosecond laser beam and a Q-switched Nd:YAG laser beam.

9. The method of claim 1, further comprising maintaining the laser beam at a power level insufficient to photodisrupt tissue during the directing, advancing and determining.

10. The method of claim 1, further comprising:
initiating laser treatment of the structure by the laser beam at the focus after determining that the focus is at or near the structure.

11. The method of claim 10, wherein initiating laser treatment comprises increasing a power level of the laser beam to a level sufficient to photodisrupt tissue.

12. A system for locating a structure in an irido-corneal angle of an eye, the system comprising:
a focusing objective configured to be coupled to the eye and aligned to receive a laser beam and to direct the laser beam toward the irido-corneal angle of the eye;
a laser subsystem configured to output the laser beam;
a second harmonic light detection apparatus configured to detect a spot of second harmonic light generated by an encounter between a focus of the laser beam and tissue; and
a control system coupled to the laser subsystem and the second harmonic light detection apparatus and configured to:
advance the focus of the laser beam to a location in the irido-corneal angle at or near the structure, and
determine that the focus is at or near the structure based on changes in an intensity of the spot of second harmonic light.

13. The system of claim 12, wherein the control system determines that the focus is at or near the structure by being further configured to:
detect a plurality of intensity states of the spot of second harmonic light as the focus of the laser beam is advanced, wherein each of the plurality of intensity states corresponds to one of a presence of the spot of second harmonic light or an absence of the spot of second harmonic light; and
detect a pattern of the plurality of intensity states that maps to the structure.

14. The system of claim 13, wherein the structure comprises a proximal surface of a trabecular meshwork and the detected pattern corresponds to an absence of the spot of second harmonic light followed by a presence of the spot of second harmonic light.

15. The system of claim 13, wherein the structure comprises an inner wall of a Schlemm's canal and the detected pattern corresponds to an absence of the spot of second harmonic light followed by a presence of the spot of second harmonic light followed by an absence of the spot of second harmonic light.

16. The system of claim 13, wherein the structure comprises an outer wall of a Schlemm's canal and the detected pattern corresponds to an absence of the spot of second harmonic light followed by a presence of the spot of second harmonic light followed by an absence of the spot of second harmonic light followed by a presence of the spot of second harmonic light.

17. The system of claim 13, wherein the structure comprises an interior of a Schlemm's canal and the detected pattern corresponds to an absence of the spot of second harmonic light followed by a presence of the spot of second harmonic light followed by an absence of the spot of second harmonic light.

18. The system of claim 12, wherein the laser beam is characterized by a first wavelength outside a visible spectrum and the spot of second harmonic light is characterized by a second wavelength within the visible spectrum.

19. The system of claim 18, wherein the first wavelength corresponds to infrared light and the second wavelength corresponds to green light.

20. The system of claim 18, wherein the second harmonic light detection apparatus comprises a camera and a filter coupled to the camera, wherein the filter is configured to detect the spot of second harmonic light.

21. The system of claim 18, wherein the second harmonic light detection apparatus comprises a photodetector and a filter coupled to the photodetector, wherein the filter is configured to detect the spot of second harmonic light.

22. The system of claim 12, wherein the laser beam is characterized by pulses having a duration of 1 nanosecond or less.

23. The system of claim 22, wherein the laser beam corresponds to one of a femtosecond laser beam and a Q-switched Nd:YAG laser beam.

24. The system of claim 12, wherein the laser subsystem is configured to maintain the laser beam at a power level insufficient to photodisrupt tissue while the laser subsystem advances the focus.

25. The system of claim 12, wherein the laser subsystem is configured to initiate laser treatment of the structure by the laser beam at the focus after the focus is determined to be at or near the structure.

26. The system of claim 25, wherein the laser subsystem is configured to initiate laser treatment of the structure by being further configured to increase a power level of the laser beam to a level sufficient to photodisrupt tissue.
